(19) United States
(12) Patent Application Publication
(10) Pub. No.: US 2024/0216348 A1
(43) Pub. Date: Jul. 4, 2024

(54) TREATMENT OF POST-OPERATIVE PAIN VIA SCIATIC NERVE BLOCK WITH SUSTAINED-RELEASE LIPOSOMAL ANESTHETIC COMPOSITIONS

(71) Applicant: Pacira Pharmaceuticals, Inc., Parsippany, NJ (US)

(72) Inventors: Roy Winston, Parsippany, NJ (US); Mary DiGiorgi, Parsippany, NJ (US)

(21) Appl. No.: 18/422,855

(22) Filed: Jan. 25, 2024

Related U.S. Application Data
(63) Continuation of application No. 18/104,970, filed on Feb. 2, 2023, now Pat. No. 11,918,565.
(60) Provisional application No. 63/422,182, filed on Nov. 3, 2022.

(51) Int. Cl. A61K 31/44; A61K 9/00; A61K 9/48; A61K 31/661; A61K 47/54

(52) U.S. Cl. CPC A61K 31/44; A61K 9/0019; A61K 9/4833; A61K 31/661; A61K 47/543

(57) ABSTRACT

Provided herein are methods of administering to a peroneal and a tibial nerve of a patient, wherein the methods include: (a) selecting an entry point of an injection needle in a leg of a patient; (b) inserting the injection needle into the patient at the entry point; (c) administering to a sciatic nerve of the patient via the injection needle saline and a pharmaceutical composition; wherein the pharmaceutical composition comprises multivesicular liposomes comprising: at least one amphipathic lipid, at least one neutral lipid, and bupivacaine phosphate, wherein the bupivacaine phosphate is encapsulated within the multivesicular liposome.

### CROSS-REFERENCE TO RELATED APPLICATIONS

This application is a continuation of U.S. application Ser. No. 18/104,970, filed Feb. 2, 2023, which claims the benefit of U.S. Provisional Application No. 63/422,182, filed Nov. 3, 2022, which are incorporated by reference herein in their entirety.

### BACKGROUND

Postsurgical pain is one of the most common forms of acute pain, which is the normal physiological response to tissue insult or injury and has adaptive value by serving as a warning of danger or damage. Most acute pain is either treatable or avoidable, especially when it occurs in a clinical setting. However, if acute pain is poorly or inappropriately treated, it may progress to chronic pain. Thus, effective postsurgical pain control is a critical element in patient recovery following surgery, as the majority of patients may experience significant pain, particularly in the first few days. Improved postsurgical pain management contributes to better healing, faster patient mobilization, shortened hospital stays, and reduced healthcare costs.

Bunionectomy is often used to manage pain in the foot from bunions. Opioids are commonly used to manage post-operative orthopedic pain. Opioid use carries a risk of developing tolerance and dependence. Thus, one goal following surgery is to improve analgesia while reducing opioid consumption.

Multimodal pain management approaches are recommended by professional societies to improve analgesia, reduce opioid use, and decrease opioid-related adverse events (AEs) following knee surgery. It is recommended that protocols include long-acting neuraxial opioids together with scheduled acetaminophen and nonsteroidal anti-inflammatory drugs (NSAIDs). However, many orthopedic pain patients still request opioids after cesarean delivery for breakthrough pain. Thus, there continues to be a need for methods of treating pain associated with surgery, including knee surgery in a subject.

### SUMMARY

Provided herein are methods of administering to a peroneal and a tibial nerve of a patient a pharmaceutical composition for post-operative analgesia, comprising: (a) selecting an entry point of an injection needle in a leg of a patient; (b) inserting the injection needle into the patient at the entry point; (c) administering to a sciatic nerve of the patient via the injection needle saline and a pharmaceutical composition; wherein the pharmaceutical composition comprises multivesicular liposomes comprising: at least one amphipathic lipid, at least one neutral lipid, and bupivacaine phosphate, wherein the bupivacaine phosphate is encapsulated within the multivesicular liposome, thereby administering to the peroneal and the tibial nerve of the patient the pharmaceutical composition for post-operative analgesia.

Provided herein are methods of administering to a peroneal and a tibial nerve of a human patient a pharmaceutical composition for post-operative analgesia, comprising: (a) selecting an entry point of an injection needle in a leg in a patient; (b) advancing a needle tip of the injection needle within a region where the sciatic nerve splits into the peroneal and tibial nerves of the patient along a trajectory that extends between the entry point and where the sciatic nerve splits into the peroneal and tibial nerves; (c) administering to said region through the injection needle saline and a therapeutically effective amount of a pharmaceutical composition; wherein the pharmaceutical composition comprises multivesicular liposomes comprising: at least one amphipathic lipid, at least one neutral lipid, and bupivacaine phosphate, wherein the bupivacaine phosphate is encapsulated within the multivesicular liposome, thereby administering to the peroneal and the tibial nerve of the human patient the pharmaceutical composition for post-operative analgesia.

Provided herein are methods of treating post-operative foot pain in a patient, comprising: (a) selecting an entry point of an injection needle in a leg of the patient; (b) inserting the injection needle into the leg of the patient at the entry point; (c) administering to the patient saline and a pharmaceutical composition within a sciatic nerve sheath; wherein the pharmaceutical composition comprises multivesicular liposomes comprising: at least one amphipathic lipid, at least one neutral lipid, and bupivacaine phosphate, wherein the bupivacaine phosphate is encapsulated within the multivesicular liposome, thereby treating post-operative foot pain in the patient.

Provided herein are methods of administering a peroneal and a tibial nerve block to a patient to reduce post-operative foot pain, comprising: (a) selecting an entry point of an injection needle in a leg of a patient, wherein the entry point comprises the lateral thigh; (b) advancing a needle tip of the injection needle within a region where the sciatic nerve splits into the peroneal and tibial nerves of the patient along a trajectory that extends between the entry point and the region where the sciatic nerve splits into the peroneal and tibial nerves; (c) piercing with the needle tip of the injection needle the sciatic nerve sheath in said region; (d) administering through the injection needle saline and a therapeutically effective amount of a multivesicular liposome pharmaceutical composition; wherein the multivesicular liposome pharmaceutical composition comprises: bupivacaine or a salt thereof; phosphoric acid; a lipid component comprising at least one amphipathic lipid and at least one neutral lipid lacking a hydrophilic head group; and, optionally, a cholesterol and/or a plant sterol, thereby administering a peroneal and the tibial nerve block to the patient.

Further, in some embodiments, the injection needle is connected to a peripheral nerve stimulator (PNS). In some embodiments, the PNS is tuned to 2 hertz and between 0.5 to 1.0 mA. In some embodiments, the PNS is used to identify the region where the sciatic nerve splits into the peroneal and tibial nerves. In some embodiments, selecting the entry point of the injection needle comprises the lateral thigh. In some embodiments, the injection needle is a 100 mm, 21-gauge needle. In some embodiments, the injection needle is insulated. In some embodiments, inserting the injection needle into the leg of the patient comprises advancing a needle tip of the injection needle a region where the sciatic nerve splits into the peroneal and tibial nerves of the patient along a trajectory that extends between the entry point and the region where the sciatic nerve splits into the peroneal and tibial nerves. In some embodiments, the insertion of the injection needle into the leg of the patient comprises piercing the sciatic nerve sheath. In some embodiments, the saline injection comprises no more than 1 to 2 mL of saline. In some embodiments, a syringe used for the saline injection is different from a syringe used for the pharmaceutical administration. In some embodiments, the method comprises administering about 30 mL of the pharmaceutical composition. In some embodiments, the multivesicular liposomes comprise: bupivacaine or a salt thereof; phosphoric acid; a lipid component comprising at least one amphipathic lipid and at least one neutral lipid lacking a hydrophilic head group; and, optionally, a cholesterol and/or a plant sterol, wherein said multivesicular liposomes are made by a process comprising: (a) preparing a first aqueous component comprising phosphoric acid; (b) preparing a lipid component comprising at least one organic solvent, at least one amphipathic lipid, and at least one neutral lipid lacking a hydrophilic head group; (c) mixing said first aqueous component and said lipid component to form a water-in-oil emulsion, wherein at least one component comprises bupivacaine or a salt thereof; (d) mixing said water-in-oil emulsion with a second aqueous component to form solvent spherules; and (e) removing the organic solvent from the solvent spherules to form multivesicular liposomes encapsulating bupivacaine phosphate. In some embodiments, the methods further comprise administering a Mayo Field Block encircling the entire metatarsal bone. In some embodiments, the Mayo Field Block comprises bupivacaine HCl.

### BRIEF DESCRIPTION OF THE DRAWINGS

FIG. 1 shows Table 1, a summary of the assessments performed in clinical trial #1.

FIG. 2 shows Table 2, a summary of the pharmacokinetic and pharmacodynamics assessments of clinical trial #1.

FIG. 3 shows Table 3, a summary of the demographics of the participants of clinical trial #1.

FIG. 4 shows Table 4, a summary of the baseline characteristics of the participants of clinical trial #1.

FIG. 5 shows Table 5, a summary of the area under the curve (AUC) of the numeric rating scale (NRS) pain intensity score at 0-96 hours post-surgery for participants in clinical trial #1.

FIG. 6 shows Table 6, a summary of the AUC of NRS pain intensity by time interval for participants in clinical trial #1.

FIG. 7 shows Table 7, a summary of total opioid consumption by time interval for participants in clinical trial #1.

FIG. 8 shows Table 8, a summary of total post-surgical opioid consumption by time interval for participants in clinical trial #1.

FIG. 9 shows Table 9, a summary of post-surgical opioid-free subjects through 96 hours for participants in clinical trial #1.

FIG. 10 shows Table 10, a summary of time to first opioid consumption post-surgery for participants in clinical trial #1.

FIG. 11 shows Table 11, a summary of the worst and average numeric rating scale pain intensity scores over the last 24 hour from post-surgery day 1 through day 4.

FIG. 12 shows Table 12, a summary of the worst and average numeric rating scale pain intensity scores over the last 24 hour from post-surgery day 1 through day 4 for Parts A+B.

FIG. 13 shows a graph where from 30 to 96 h post-surgery (Part A+B), the EXP133 arm had a significantly lower mean current pain intensity than the BUP50 arm (p-values <0.00008).

FIG. 14 shows Table 13, a summary of the pharmacokinetic parameters.

FIG. 15 shows Table 14, a summary of the time to onset and duration of motor and sensory block for participants in clinical trial #1.

FIGS. 16A-B show FIG. 2, a KM plot of time to onset (FIG. 16A) and duration (FIG. 16B) of motor block for participants in clinical trial #1.

FIGS. 17A-B show FIG. 3, a KM plot of time to onset (FIG. 17A) and duration (FIG. 17B) of sensory nerve block for participants in clinical trial #1.

FIG. 18 shows Table 15, a summary of treatment-emergent adverse events for participants in clinical trial #1.

FIG. 19 shows Table 16, a summary of treatment-emergent adverse events occurring in ≥5% of subjects in any treatment arm.

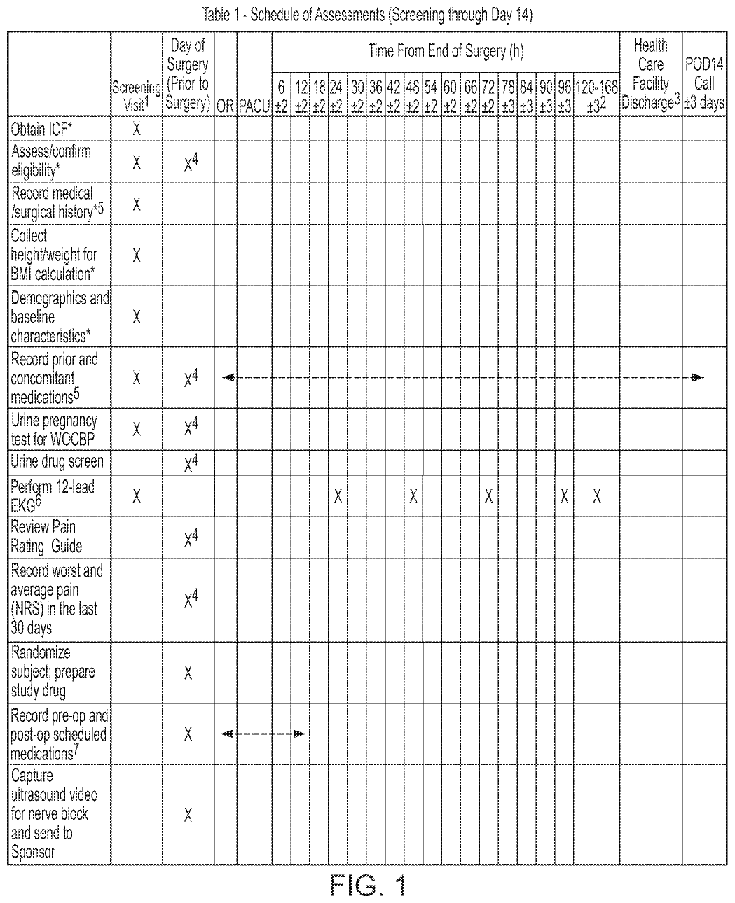

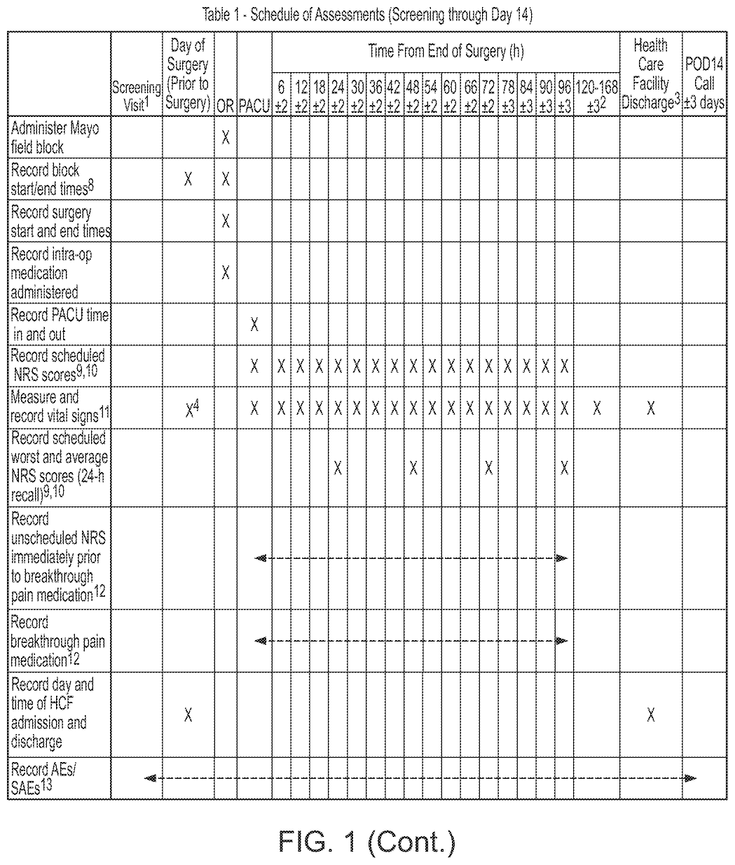

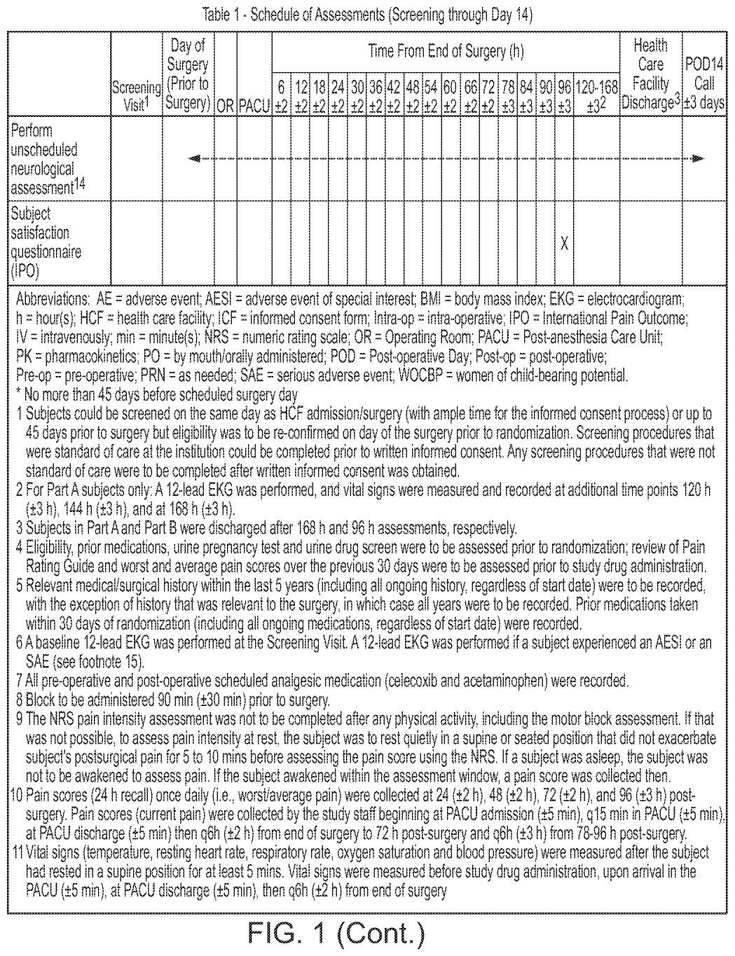

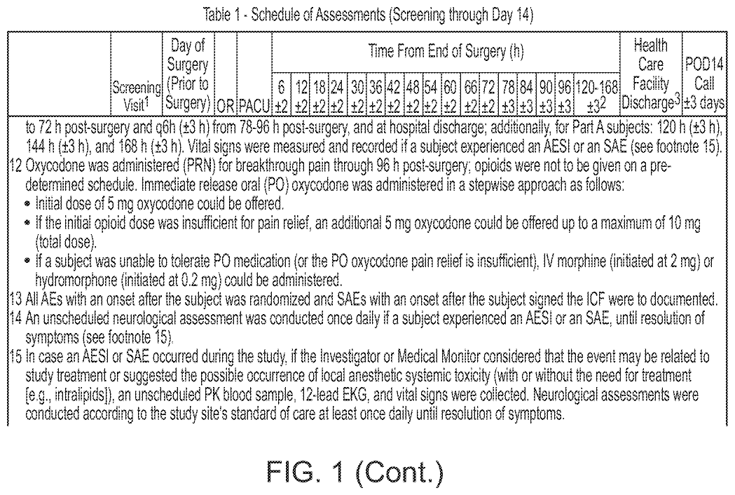

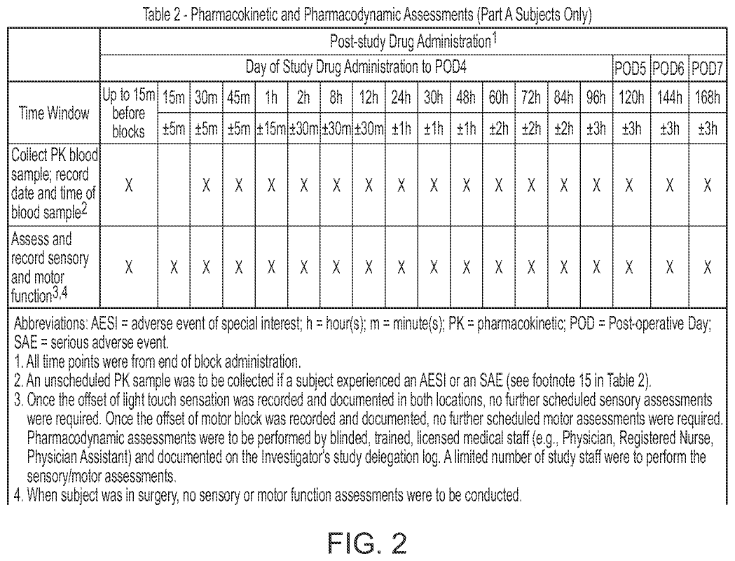

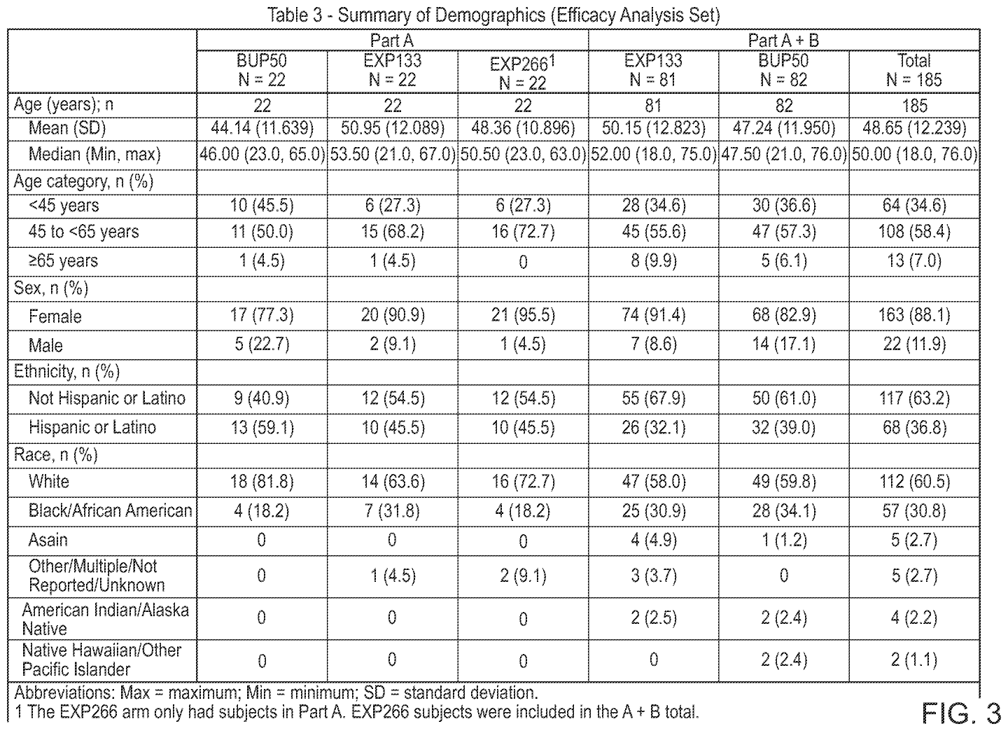

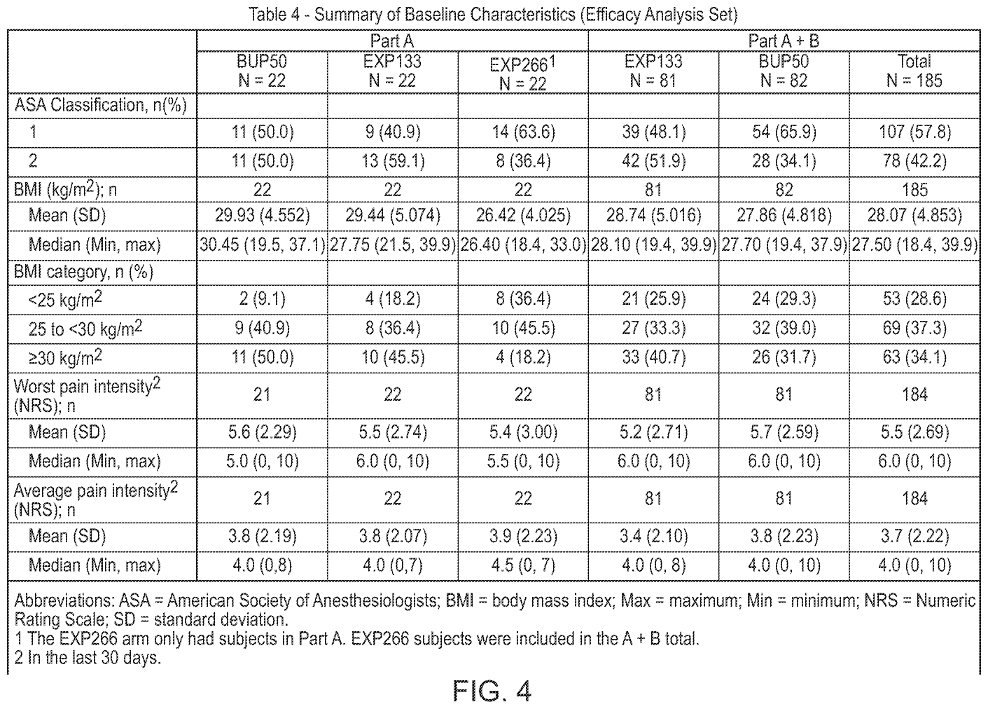

### DETAILED DESCRIPTION

Unless otherwise defined, all technical and scientific terms used herein have the same meaning as commonly understood by one of ordinary skill in the art to which this disclosure belongs. Although methods and materials similar to those described herein can be used in the practice or testing of the present disclosure, suitable methods and materials are described below. All publications, patent applications, patents, and other references mentioned herein are incorporated by reference in their entirety. In addition, the materials, methods, and examples are illustrative only and not intended to be limiting.

As used herein, the term “subject” refers to any animal (e.g., a mammal), including, but not limited to, humans, non-human primates, rodents, and the like, which is to be the recipient of a particular treatment. Typically, the terms “subject” and “patient” are used interchangeably herein in reference to a human subject.

As used herein, the term “pain” means a physiologic and/or psychologic reaction or response to potential or actual stimulus that may result in tissue damage, injury, disease, or other condition(s). Types of pain include but are not limited to acute pain, chronic pain, thermal pain, traumatic pain, chemical pain, inflammatory pain, ischemic pain, blunt pain, sharp pain, prickling pain, visceral pain, and neuropathic pain.

### Sciatic Nerve Block at the Popliteal Fossa

In some embodiments, the methods include administering a pharmaceutical composition to the sciatic nerve. In some embodiments, the pharmaceutical composition is administered to the sciatic nerve at the level of the popliteal fossa. In some embodiments, the method includes administering a pharmaceutical composition to a peroneal and/or a tibial nerve. In some embodiments, the method includes administering a pharmaceutical composition to a peroneal and a tibial nerve. The sciatic nerve can be described a long and thick nerve made up of five nerve roots, two from the lower back region called the lumbar spine and three from the final section of the spine called the sacrum. The five nerve roots come together to form a right and left sciatic nerve. One sciatic nerve runs through each side of the body, through the hips, buttocks and down a leg, ending just below the knee. There, the sciatic nerve branches into other nerves, which continue down the leg and into the foot and toes. The sciatic nerve bifurcates into the tibial (medial) and common peroneal (lateral) nerves at approximately 6 to 10 cm proximal to the popliteal crease, though there is significant anatomic variation in the location of the split. A common sheath envelops these two nerves from their origin in the pelvis, which is distinctly separate from the epineurium of each nerve. Ultrasound imaging has shown that injecting local anesthetic within this sheath consistently gives a rapid onset, safe, and effective block. As the sciatic nerve descends toward the knee, the two components eventually diverge just proximal to the popliteal fossa, giving rise to the tibial and common peroneal nerves. The nerves traverse the diamond-shaped popliteal fossa, which is bounded laterally by the biceps femoris tendon and medially by the semitendinosus and semimembranosus tendons. The division of the sciatic nerve usually occurs between 50 and 120 mm proximal to the popliteal fossa crease. Following its divergence from the sciatic nerve, the peroneal nerve continues its path laterally and descends along the head and neck of the fibula. Its major branches in this region are branches to the knee joint and cutaneous branches that form the sural nerve. Its terminal branches are the superficial and deep peroneal nerves. The tibial nerve is the larger of the two divisions of the sciatic nerve and continues its path vertically through the popliteal fossa. Its terminal branches are the medial and lateral plantar nerves. Its collateral branches give rise to the cutaneous sural nerves, muscular branches to the muscles to the calf, and articular branches to the ankle joint.

In some embodiments, administering a pharmaceutical composition to the sciatic nerve includes a sciatic nerve block. In some embodiments, administering a pharmaceutical composition to the sciatic nerve, at the level of the popliteal fossa, includes a popliteal block. In some embodiments, administering a pharmaceutical composition to the sciatic nerve is an injection into the sheath of the sciatic nerve. In some embodiments, a sciatic nerve block can be used to deliver sensory anesthesia for procedures involving the lower leg and/or foot (e.g., calf, tibia, fibula, ankle, and foot). In some embodiments, a sciatic nerve block can be used to deliver sensory anesthesia for distal branches of the peroneal and/or tibial nerves. In some embodiments, administering a pharmaceutical composition to the sciatic nerve can be the administration of a sensory nerve block and/or a motor nerve block. In some embodiments, the sciatic nerve can be accessed through the lateral thigh.

In some embodiments, the sciatic nerve and/or popliteal fossa can be located in the patient leg by way of surface landmarks. In some embodiments, the sciatic nerve and/or popliteal fossa can be located by way of ultrasound guidance. In some embodiments, the sciatic nerve and/or popliteal fossa can be located a combination of surface landmarks and ultrasound guidance.

### Pharmaceutical Compositions

Provided herein are analgesic pharmaceutical compositions. In some embodiments the pharmaceutical compositions are useful for the amelioration of post-operative analgesic pain.

In some embodiments, the pharmaceutical compositions include multivesicular liposomes. Multivesicular liposomes (or “MVL”, which is used herein to refer to a multivesicular liposome or a plurality of multivesicular liposomes) are lipid vesicles having multiple non-concentric internal aqueous chambers having internal membranes distributed as a network throughout the MVL. The chambers may contain acids which are effective to enable the encapsulation of bupivacaine or a salt thereof and to modulate its release rate. A preparation of MVL is described, for example, in Kim et al., Biochim. Biophys. Acta 728, 339-348, 1983. In some embodiments, a MVL is prepared in accordance with a process as described in U.S. Pat. No. 9,192,575, incorporated by reference herein in its entirety. In some embodiments, a MVL is prepared in accordance with a process as described in U.S. Pat. No. 8,182,835, incorporated by reference herein in its entirety. In some embodiments, a MVL is prepared in accordance with a process as described in U.S. Pat. No. 8,834,921, incorporated by reference herein in its entirety. In some embodiments, a MVL is prepared in accordance with a process as described in U.S. Pat. No. 9,205,052, incorporated by reference herein in its entirety.

In some embodiments the multivesicular liposomes (“MVL”) are made by the following process. A “water-in-oil” type emulsion containing a non-hydrohalic acid salt of bupivacaine, such as bupivacaine phosphate, is formed from two immiscible phases, a lipid phase and a first aqueous phase. The lipid phase is made up of at least one amphipathic lipid and at least one neutral lipid in a volatile organic solvent. The term “amphipathic lipid” refers to molecules having a hydrophilic “head” group and a hydrophobic “tail” group and may have membrane-forming capability. As used herein, amphipathic lipids include those having a net negative charge, a net positive charge, and zwitterionic lipids (having no net charge at their isoelectric point). The term “neutral lipid” refers to oils or fats that have no vesicle-forming capability by themselves, and lack a charged or hydrophilic “head” group. Examples of neutral lipids include, but are not limited to, glycerol esters, glycol esters, tocopherol esters, sterol esters which lack a charged or hydrophilic “head” group, and alkanes and squalenes.

The amphipathic lipid is chosen from a wide range of lipids having a hydrophobic region and a hydrophilic region in the same molecule. Suitable amphipathic lipids are zwitterionic phospholipids, including phosphatidylcholines, phosphatidylethanolamines, sphingomyelins, lysophosphatidylcholines, and lysophosphatidylethanolamines. Also suitable are the anionic amphipathic phospholipids such as phosphatidylglycerols, phosphatidylserines, phosphatidylinositols, phosphatidic acids, and cardiolipins. Also suitable are the cationic amphipathic lipids such as acyl trimethylammonium propanes, diacyl dimethylammonium propanes, and stearylamines.

Suitable neutral lipids are triglycerides, propylene glycol esters, ethylene glycol esters, and squalene. Examples of triglycerides useful in the present disclosure are triolein, tripalmitolein, trimyristolein, trilinolein, tributyrin, tricaproin, tricaprylin, and tricaprin. The fatty chains in the triglycerides useful in the present disclosure can be all the same, or not all the same (mixed chain triglycerides), including all different. Both saturated and unsaturated fatty chains are useful in the present disclosure. The propylene glycol esters can be mixed diesters of caprylic and capric acids.

Many types of volatile organic solvents can be used in the present disclosure, including ethers, esters, halogenated ethers, hydrocarbons, halohydrocarbons, or Freons. For example, diethyl ether, chloroform, tetrahydrofuran, ethyl acetate, Forane, and any combinations thereof are suitable for use in making the compositions of the present disclosure.

Optionally, other components are included in the lipid phase. Among these are cholesterol or plant sterols.

The first aqueous phase includes bupivacaine or a salt thereof, such as bupivacaine phosphate, at least one polyhydroxy carboxylic acid, and at least one di- or tri-protic mineral acid. In some embodiments, also included is hydrochloric acid. The di- or tri-protic mineral acids include sulfuric acid, and phosphoric acid. Also included in the first aqueous phase are such polyhydroxy carboxylic acids as glucuronic acid, gluconic acid, and tartaric acid. The di- and tri-protic mineral acids and the polyhydroxy organic acids are present in the first aqueous phase in concentrations of from 0.01 mM to about 0.5 M, or preferably from about 5 mM to about 300 mM. When hydrochloric acid is used, it is present in lower amounts, from about 0.1 mM to about 50 mM, or preferably from about 0.5 mM to about 25 mM.

The lipid phase and first aqueous phase are mixed by mechanical turbulence, such as through use of rotating or vibrating blades, shaking, extrusion through baffled structures or porous pipes, by ultrasound, or by nozzle atomization, to produce a water-in-oil emulsion. Thus, bupivacaine or a salt thereof, such as bupivacaine phosphate, is encapsulated directly in the first step of MVL manufacture.

The whole water-in-oil emulsion is then dispersed into a second aqueous phase by means described above, to form solvent spherules suspended in the second aqueous phase. The term “solvent spherules” refers to a microscopic spheroid droplet of organic solvent, within which are suspended multiple smaller droplets of aqueous solution. The resulting solvent spherules therefore contain multiple aqueous droplets with the bupivacaine or a salt thereof, such as bupivacaine phosphate, dissolved therein. The second aqueous phase can contain additional components such as glucose, and/or lysine.

The volatile organic solvent is then removed from the spherules, for instance by surface evaporation from the suspension: When the solvent is substantially or completely evaporated, MVL are formed. Gases which can be used for the evaporation include nitrogen, argon, helium, oxygen, hydrogen, and carbon dioxide. Alternatively, the volatile solvent can be removed by sparging, rotary evaporation, or with the use of solvent selective membranes.

In some embodiments, an MVL is prepared in accordance with a process as described in U.S. Pat. No. 10,398,648, incorporated by reference herein in its entirety. In some embodiments, a MVL is prepared in accordance with a process as described in U.S. Pat. No. 9,585,838 incorporated by reference herein in its entirety.

In some embodiments, an MVL is prepared in accordance with a process as described in US Published Patent Applications US 2011-0250264, US 2013-0306759, US 2013-0177634, US 2013-0177633, US 2013-0177635, US 2013-0195965, US 2013-0177636, US 2013-0183373, US 2013-0177638, US 2013-0177637, US 2013-0183372, US 2013-0183375, US 2016-0361260 or US 2018-0092847, each of which is incorporated by reference herein in its entirety.

In some embodiments, an MVL is prepared in accordance with a process as described in U.S. Pat. Nos. 11,033,495; 11,179,336; 11,278,494; 11,304,904; 11,311,486; 11,357,727; 11,426,348; 11,452,691, each of which is incorporated by reference herein in its entirety.

In some embodiments, the pharmaceutical compositions described herein can be combined, used in conjunction with, or used in an anesthetic or analgesic program with other anesthetics or analgesics.

Examples of anesthetics, include but are not limited to, propofol, etomidate, methohexital and sodium thiopental, midazolam, diazepam, and ketamine, benzocaine, chloroprocaine, cocaine, cyclomethycaine, dimethocaine, propoxycaine, procaine, proparacaine, tetracaine, articaine, bupivacaine, carticaine, dibucaine, etidocaine, levobupivacaine, lidocaine, mepivacaine, piperocaine, prilocaine, ropivacaine, trimecaine, saxitoxin, and tetrodotoxin. Examples of amide anesthetics, include but are not limited to, articaine, bupivacaine, carticaine, dibucaine, etidocaine, levobupivacaine, lidocaine, mepivacaine, piperocaine, prilocaine, ropivacaine, and trimecaine. In some embodiments, the multivesicular liposomes further comprise bupivacaine, morphine, cytarabine, or their pharmaceutically acceptable salts as the therapeutic agent. In some embodiments, the multivesicular liposomes further comprise bupivacaine phosphate, morphine sulfate, or cytarabine HCl.

The term “therapeutically effective” as it pertains to bupivacaine or a salt thereof, such as bupivacaine phosphate, present in the pharmaceutical compositions described herein, means that an anesthetic present in the first aqueous phase within the multivesicular liposome is released in a manner sufficient to achieve a particular level of anesthesia. Exact dosages will vary depending on such factors as the particular anesthetic, as well as patient factors such as age, sex, general condition, patient size, and the like. Those of skill in the art can readily take these factors into account and use them to establish effective therapeutic concentrations without resort to undue experimentation.

As used herein, “non-liposomal bupivacaine” refers to bupivacaine or a salt thereof that is not in liposomal form. For example, “non-liposomal bupivacaine” refers to bupivacaine or a salt thereof that is not comprised in a multivesicular liposome. The term “non-liposomal bupivacaine” encompasses compositions comprising bupivacaine, or a salt thereof, that is not in liposomal form.

Examples of analgesics can include opioid analgesics and non-opioid analgesics. Non-limiting examples of opioid analgesics include hydrocodone, oxycodone, propoxyphene, or fentanyl, thiosemicarbazone, p-nitrophenylhydrazone, o-methyloxime, semicarbazone, or bis (methylcarbamate), oxycodone, a pharmaceutically acceptable salt or its thiosemicarbazone, p-nitrophenylhydrazone, o-methyloxime, semicarbazone, or bis-methylcarbamate. Non-limiting examples of non-opioid analgesics useful in the present invention include aspirin; acetaminophen; a non-steroidal anti-inflammatory drug (NSAID), an arylalkanoic acid, a profen, a fenamic acid, an oxicam, a pyrazolidine derivative; a Cox-2 inhibitor, a local analgesic, an anti-depressant, an atypical analgesic, a psychotropic agent, an NMDA receptor antagonist, an α2-adrenoreceptor agonists and a synthetic drug having narcotic properties.

Embodiments of the present disclosure also include compositions prepared for storage or administration that include a pharmaceutically effective amount of the desired compounds in a pharmaceutically acceptable carrier or diluent. Acceptable carriers or diluents for therapeutic use are well known in the pharmaceutical art, and are described, for example, in Remington's Pharmaceutical Sciences, Academic Press, (Adeboye Adejareedit edit., 2020), hereby incorporated by reference in its entirety.

A pharmaceutically effective dose is that dose required to prevent, inhibit the occurrence, or treat (alleviate a symptom to some extent, preferably all of the symptoms) pain. One skilled in the art appreciates that compositions and methods of the present disclosure can be used to treat multiple types of pain, and that the effective dose may be different for different types of pain.

Types of pain include but are not limited to thermal pain, chemical pain, inflammatory pain, ischemic pain, traumatic pain, blunt pain, sharp pain, prickling pain, and visceral pain. The pharmaceutically effective dose depends on the type of condition (e.g., pain), the composition used, the route of administration, the type of mammal being treated, the physical characteristics of the specific mammal under consideration (including but not limited to age, physical condition, surgical or other medical procedures being performed, circulatory capacity, cardiovascular function, pain tolerance, nerve function, liver function), concurrent medication, and other factors that those skilled in the medical arts will recognize.

### Methods of Administering

Provided herein are methods of administering to a peroneal and a tibial nerve of a patient a pharmaceutical composition for post-operative analgesia, comprising: (a) selecting an entry point of an injection needle in a leg of a patient; (b) inserting the injection needle into the patient at the entry point; (c) administering to a sciatic nerve of the patient via the injection needle saline and a pharmaceutical composition; wherein the pharmaceutical composition comprises multivesicular liposomes comprising: at least one amphipathic lipid, at least one neutral lipid, and bupivacaine phosphate, wherein the bupivacaine phosphate is encapsulated within the multivesicular liposome, thereby administering to the peroneal and the tibial nerve of the patient the pharmaceutical composition for post-operative analgesia.

Provided herein are methods of administering to a peroneal and a tibial nerve of a human patient a pharmaceutical composition for post-operative analgesia, comprising: (a) selecting an entry point of an injection needle in a leg in a patient; (b) advancing a needle tip of the injection needle within a region where the sciatic nerve splits into the peroneal and tibial nerves of the patient along a trajectory that extends between the entry point and where the sciatic nerve splits into the peroneal and tibial nerves; (c) administering to said region through the injection needle saline and a therapeutically effective amount of a pharmaceutical composition; wherein the pharmaceutical composition comprises multivesicular liposomes comprising: at least one amphipathic lipid, at least one neutral lipid, and bupivacaine phosphate, wherein the bupivacaine phosphate is encapsulated within the multivesicular liposome, thereby administering to the peroneal and the tibial nerve of the human patient the pharmaceutical composition for post-operative analgesia.

Provided herein are methods of treating post-operative foot pain in a patient, comprising: (a) selecting an entry point of an injection needle in a leg of the patient; (b) inserting the injection needle into the leg of the patient at the entry point; (c) administering to the patient saline and a pharmaceutical composition within a sciatic nerve sheath; wherein the pharmaceutical composition comprises multivesicular liposomes comprising: at least one amphipathic lipid, at least one neutral lipid, and bupivacaine phosphate, wherein the bupivacaine phosphate is encapsulated within the multivesicular liposome, thereby treating post-operative foot pain in the patient.

Provided herein are methods of administering a peroneal and a tibial nerve block to a patient to reduce post-operative foot pain, comprising: (a) selecting an entry point of an injection needle in a leg of a patient, wherein the entry point comprises the lateral thigh; (b) advancing a needle tip of the injection needle within a region where the sciatic nerve splits into the peroneal and tibial nerves of the patient along a trajectory that extends between the entry point and the region where the sciatic nerve splits into the peroneal and tibial nerves; (c) piercing with the needle tip of the injection needle the sciatic nerve sheath in said region; (d) administering through the injection needle saline and a therapeutically effective amount of a multivesicular liposome pharmaceutical composition; wherein the multivesicular liposome pharmaceutical composition comprises: bupivacaine or a salt thereof; phosphoric acid; a lipid component comprising at least one amphipathic lipid and at least one neutral lipid lacking a hydrophilic head group; and, optionally, a cholesterol and/or a plant sterol, thereby administering a peroneal and the tibial nerve block to the patient.

In some embodiments, the pharmaceutical composition is administered around or within a volume of 30 ml.

In some embodiments, the efficacy of a lower dose of MVL exceeds the efficacy of a higher dose of MVL. In some embodiments, a lower dose of MVL is between 100 and 150 mg MVL. In some embodiments, a higher dose of MVL is between 250 and 300 mg. In some embodiments, efficacy is defined as understood in the art. In some embodiments, efficacy is an improvement in any of the outcomes monitored in the studies described herein. Having a lower dose of MVL be more efficacious than a higher dose of MVL was surprising because it was expected that higher doses of MVL would produce a greater effect.

In some embodiments, the methods of the disclosure produce unexpectedly good anesthesia such that fewer rescue pain medications are needed to manage post-operative pain. In some embodiments, the methods of the disclosure produce unexpectedly good anesthesia such that a multimodal analgesic regimen (a non-opioid analgesic followed by an opioid analgesic) is not needed for post-operative pain rescue. In some embodiments, the methods of the disclosure produce unexpectedly good anesthesia such that much less or no opioids are needed for post-operative pain rescue. In some embodiments, the methods of the disclosure produce unexpectedly good anesthesia such that much less or no non-opioid medication is needed for post-operative pain rescue. In some embodiments, the methods of the disclosure produce unexpectedly good anesthesia such that only non-opioid medications are needed for post-operative pain rescue. In some embodiments, the methods of the disclosure produce unexpectedly good anesthesia such that only opioid medications are needed for post-operative pain rescue.

### Routes of Administration

Anesthetics of the present disclosure may be delivered regionally or locally. “Regional” or “local” anesthesia, as used herein, is distinct from general anesthesia and refers to anesthetic procedures which allow for the preferential delivery of an anesthetic to a specific region of the body, such as near a nerve or a nerve bundle. In contrast, general anesthesia allows for the systemic administration of an anesthetic, e.g., via intravenous administration. Regional or local anesthesia typically allows for a lower total body concentration (although elevated local concentrations) of an anesthetic to be administered to a subject for analgesia or diminished pain perception of at least a portion of the subject's body. For example, intrathecal anesthesia, epidural anesthesia, and nerve blocks are examples of regional or local anesthesia.

A pharmacological composition can refer to a composition in a form suitable for administration, e.g., perineural administration, into a subject or proximal to at least one nerve of a subject, including for example wherein the subject is a human. Suitable forms, in part, depend upon the use or the route of entry. Examples of routes of entry include but are not limited to injection (including but not limited to subcutaneous injection), single injection, serial injection, indwelling catheter, and continuous infusion. Such routes of entry should not prevent the composition from reaching a target cell (i.e., a neuron). For example, injectable pharmacological compositions should be soluble. Other factors are known in the art, and include considerations such as toxicity and forms that prevent the composition or formulation from exerting its effect.

### EXAMPLES

### Example 1—Clinical Trial #1

A multicenter, randomized, double-blind clinical trial examining efficacy and safety of the pharmaceutical composition comprising: a) the multivesicular liposome disclosed herein and b) the aqueous phase disclosed herein, wherein the aqueous phase is encapsulated within the multivesicular liposome, was performed as described below. The pharmaceutical composition is referred to below as the “multivesicular liposomal” pharmaceutical composition, or “MVL”.

The trial compared the magnitude of the postsurgical analgesic effect following a single dose of the MVL admixed with bupivacaine HCl vs. bupivacaine HCl when administered via an sciatic nerve block in subjects undergoing bunionectomy. Further, the trial compared post-surgical opioid consumption following a single dose of EXPAREL vs. bupivacaine HCl. Additionally, the trial compared the time to first opioid consumption post-surgery, following a single dose of EXPAREL vs. bupivacaine HCl. Further, the trial assessed the safety profile of EXPAREL and bupivacaine HCl.

| Abbreviation | Definition |
|---|---|
| λ_{z} | Apparent terminal elimination rate constant |
| AE | Adverse event |
| AESI | Adverse event of special interest |
| ANCOVA | Analysis of covariance |
| ASA | American Society of Anesthesiologists |
| AUC | Area under the curve |
| AUC_{0-∞} | Area under the curve from the time of dosing to infinity |
| AUC₀₋ₗₐₛₜ | Area under the curve from the time of dosing |
| | to the of the last quantifiable concentration |
| BLOQ | Below the limit of quantification |
| BMI | Body mass index |
| CFR | Code of Federal Regulations |
| CI | Confidence interval |
| CL/F | Apparent clearance |
| Cₘₐₓ | Maximum plasma concentration |
| CMH | Cochran-Mantel-Haenszel |
| COVID-19 | Coronavirus disease 2019 |
| CV | Coefficient of variation |
| Diff | Difference |
| eCRF | Electronic case report form |
| EKG | Electrocardiogram |
| FDA | Food and Drug Administration |
| GCP | Good Clinical Practice |
| h | Hour(s) |
| Hₐ | Alternative hypothesis |
| HCF | Health care facility |
| HCl | Hydrochloride |
| ICF | Informed consent form |
| ICH | International Council for Harmonisation |
| ID | Identification |
| IEC | Independent Ethics Committee |
| Intra-op | Intra-operative |
| IPO | International Pain Outcome |
| IRB | Institutional Review Board |
| IV | Intravenous(ly) |
| KM | Kaplan-Meier |
| LLOQ | Lower limit of quantification |
| LOCF | Last observation carried forward |
| LS(M) | Least squares (mean) |
| Max | Maximum |
| MedDRA | Medical Dictionary for Regulatory Activities |
| Min | Minimum |
| min | Minute(s) |
| NC | Not computed |
| NCA | Noncompartmental analysis |
| NRS | Numeric rating scale |
| NS | Normal saline |
| NSAID | Non-steroidal anti-inflammatory drug |
| OMED | Oral morphine equivalent |
| OR | Operating Room |
| PACU | Post-anesthesia Care Unit |
| PD | Pharmacodynamic(s) |
| PK | Pharmacokinetic(s) |
| PO | Orally administered |
| POD | Post-operative day |
| Post-op | Post-operative |
| PP | Predictive power |
| Pre-op | Pre-operative |
| PRN | As needed |
| PT | Preferred term |
| Reduct | Reduction |
| SAE | Serious adverse event |
| SAP | Statistical analysis plan |
| SD | Standard deviation |
| SE | Standard error |
| SOC | System organ class |
| t_{1/2el} | Apparent terminal elimination half-life |
| TEAE | Treatment-emergent adverse event |
| Tₘₐₓ | Time to maximum plasma concentration |
| V_{d}/F | Apparent volume of distribution |
| WOCBP | Women of child-bearing potential |

### Inclusion Criteria

Each subject had to meet the following criteria to be eligible for the study:
1. Male or female, ages 18 or older at Screening.
2. American Society of Anesthesiologists (ASA) physical status 1, 2, or 3.
3. Able to provide informed consent, adhere to the study schedule, and complete all study assessments
4. Primary surgical indication was related to a bunion deformity (i.e., hallux valgus) and subject was scheduled to undergo a distal metaphyseal osteotomy procedure (e.g., Austin procedure as opposed to Lapiplasty, Lapidus bunionectomies or base wedge bunionectomies).
5. Indicated to undergo elective (i.e., not emergency) bunionectomy.
6. Body mass index (BMI) ≥18 and <40 kg/m2.

### Exclusion Criteria

Subjects who met any of the following criteria were excluded from the study:
1. Allergy, hypersensitivity, intolerance, or contraindication to any of the study medications for which an alternative was not named in the Protocol (e.g., amide-type local anesthetics, opioids, bupivacaine HCl, and non steroidal anti-inflammatory drugs [NSAIDs]).
2. Concurrent painful physical condition (e.g., arthritis, fibromyalgia, and cancer) that may have required analgesic treatment with NSAIDs or opioids in the post-dosing period for pain that was not strictly related to the foot surgery and which may have confounded the post-dosing assessments.
3. Inadequate sensory function of the foot/ankle as assessed.
4. History of, suspected, or known addiction to or abuse of illicit drug(s), prescription medicine(s), or alcohol within the past 2 years.
5. Administration of an investigational drug within 30 days or 5 elimination half-lives of such investigational drug, whichever was longer, prior to study drug administration, or planned administration of another investigational product or procedure during the subject's participation in this study.
6. Previous participation in an EXPAREL study.
7. Uncontrolled anxiety, schizophrenia, or other psychiatric disorder that could interfere with study assessments or compliance.
8. Currently pregnant, nursing, or planning to become pregnant during the study.
9. Clinically significant medical disease that would have made participation in a clinical study inappropriate. This included diabetic neuropathy, coagulation, or bleeding disorders, severe peripheral vascular disease, renal insufficiency, hepatic dysfunction, or other conditions that would have constituted a contraindication to participation in the study.
10. Currently on a neuromodulating agent (e.g., gabapentin, pregabalin [Lyrica®], duloxetine [Cymbalta®], etc.).
11. Current use of systemic glucocorticoids within 30 days of randomization in this study.
12. Use of dexmedetomidine HCl (Precedex®) or clonidine within 3 days of study drug administration.
13. Any use of marijuana (including tetrahydrocannabinol and cannabidiol) within 30 days prior to randomization, or planned use during the course of the study.
14. Chronic opioid use within 30 days prior to randomization (average ≥30 OMED/day).

### Methods

Clinical Study #1 was a Phase 3, multicenter, randomized, double-blind, active-controlled study in 180 subjects undergoing bunionectomy. Subjects received a sciatic (in the popliteal fossa) nerve block with a single dose of either EXPAREL 266 mg (EXP266), EXPAREL 133 mg (EXP133), or 0.25% bupivacaine HCl (50 mg; BUP50). The study was conducted in two parts (Part A and Part B). Part A was to be completed and analyzed before enrollment in Part B was initiated. Part A was a 3-arm cohort with 66 subjects undergoing bunionectomy to obtain information on pharmacokinetics (PK), pharmacodynamics (PD), efficacy, and safety. Subjects were randomized (1:1:1) to the EXP266, EXP133, and BUP50 treatment arms. Part B was a 2-arm cohort with 119 subjects undergoing bunionectomy to evaluate the efficacy and safety of EXPAREL compared with bupivacaine HCl. The dose of the EXPAREL arm in Part B was determined based on the interim analysis of the Part A cohort results.

Subject participation began upon obtaining informed consent, which was obtained within 45 days prior to administration of the study drug. Screening procedures included assessment of eligibility; recording of medical/surgical history, prior and concomitant medications (related to medical history), demographics and baseline characteristics, and height and weight for body mass index (BMI) calculation; assessment of chronic opioid and any cannabis use in the past 30 days (average ≥30 OMED/day); urine pregnancy test for women of childbearing potential (WOCBP); 12-lead electrocardiogram (EKG); and monitoring of adverse events (AEs) and serious adverse events (SAEs).

On the day of surgery, subjects received an ultrasound-guided sciatic (in the popliteal fossa) nerve block with one of the following treatments:
EXP266 arm: subjects randomized to this treatment arm received 20 mL (266 mg) EXPAREL mixed with 10 mL saline (Part A only; per interim analysis was not selected for Part B)
EXP133 arm: subjects randomized to this treatment arm received 10 mL (133 mg) EXPAREL mixed with 20 mL saline (Part A and B)
BUP50 arm: subjects randomized to this treatment arm received 20 mL (50 mg) 0.25% bupivacaine HCl mixed with 10 mL saline (Part A and B)

All subjects in Part A and Part B received a Mayo field block with 20 mL of 0.5% bupivacaine HCl after study drug administration.

Treatment prior to study drug administration could have included Celecoxib 200 mg orally administered within 4 hours prior to surgery. Other permitted prior medications and therapy include 1 to 2 mg of midazolam and/or ondansetron. Restricted medications and therapy prior to drug study administration included systemic glucocorticosteroids and neuromodulating agents (e.g., gabapentin, pregabalin [Lyrica®], duloxetine [Cymbalta®], etc.); no long-acting or sustained release opioid medications and NSAIDs (except for low-dose acetylsalicylic acid used for cardiovascular protection) within 3 days of study drug administration; no dexmedetomidine HCl (Precedex®) or clonidine within 3 days of study drug administration; no scopolamine patches; no opioid medications within 24 hours of study drug administration; no use of an investigational product within 30 days or 5 elimination half-lives of such investigational drug, whichever was longer, prior to study drug administration, or planned administration of another investigational product or procedure during the subject's participation in this study was not permitted; no drugs (other than the described bupivacaine HCl admixture) were to be admixed with study drug (e.g., epinephrine, dexamethasone, clonidine); no lidocaine or other local anesthetics were locally administered in the area of the nerve block administration other than use in a superficial cutaneous wheal for needle insertion.

Perioperative treatment. Perioperative treatment included receiving a Mayo field block with 20 mL 0.5% bupivacaine HCl immediately following study drug administration (was to be done with the same set-up for the nerve block). A participant could have received 1000 mg of IV acetaminophen at the time of surgical incision, single-dose administration of ondansetron or metoclopramide for nausea, or propofol for induction and intra-operative sedation. Restricted medications included no other medication (including opioids) were to be mixed with the bupivacaine for spinal anesthesia; no use of dexamethasone, acetaminophen, ketorolac, or other NSAIDs preemptively or intra operatively, except for emergency use to treat an AE; no intra operative use of opioids (except IV fentanyl at a dose not exceeding 1 μg/kg, unless deemed medically necessary) and ketamine. Neuraxial or regional anesthesias were not permitted. Lidocaine and other local anesthetics were not permitted to be locally administered in the area of the nerve block administration.

Postsurgical pain for breakthrough pain. An unscheduled pain intensity assessment using the NRS (measured as “On a scale from 0 to 10, where 0 equals no pain and 10 equals the worst possible pain, how much pain are you experiencing in your operative foot right now?”) was to be completed immediately prior to administration of any breakthrough pain medication up to 96 h post-surgery. Medications were administered PRN; opioids were not to be given on a pre-determined schedule. Immediate release PO oxycodone could be administered in a stepwise approach as follows: initial doe of 5 mg oxycodone offered; if the initial opioid dose was insufficient for pain relief, an additional 5 mg oxycodone could be offered up to a maximum of 10 mg (total dose); if a subject was unable to tolerate PO medication or the PO oxycodone pain relief was insufficient, IV morphine (initiated at 2 mg) or hydromorphone (initiated at 0.2 mg) could be administered. No NSAIDs or other opioids, including tramadol were allowed for breakthrough pain management. No acetaminophen (other than the scheduled IV acetaminophen) was to be used for breakthrough pain. Pain management modalities were standardized during the first 96 h post-surgery. After 96 h, the analgesic regimen could be adjusted for each subject individually as deemed appropriate by the physician responsible for the postsurgical care. All postsurgical analgesics administered, were to be recorded through hospital discharge.

Efficacy assessments included pain intensity scores focused on the operative foot using the Numeric Rating Scale (NRS), total postsurgical opioid consumption in OMED, time to first opioid consumption post-surgery, and percentage of opioid-free patients through 96 hours, and worst and average NRS pain intensity scores at 24 h, 48 h, 72 h, and 96 h from end of surgery. Additionally, blood samples for pharmacokinetic (PK) analysis (area under the curve [AUC], maximum plasma concentration [Cmax], time of maximum plasma concentration [Tmax], including early and late Cmax and Tmax (EXP133-ADMIX arm only), apparent terminal elimination half-life [t½el], apparent clearance [CL/F], and apparent volume of distribution [Vd/F]). Pharmacodynamic (PD) assessment data, including median time to onset of sensory block and motor block, and median duration of sensory and motor block were collected.

Safety assessments included monitoring of AEs from the time of randomization through POD 14. Safety endpoints include incidence of treatment-emergent AEs and SAEs from the start of the nerve block procedure through POD 14.

### End Points

The primary endpoint was the AUC of the NRS pain intensity scores from 0 to 96 hours post-surgery.

Secondary efficacy endpoints included total postsurgical opioid consumption in OMED from 0 to 96 hours post-surgery; percentage of opioid-free subjects through 96 hours; time to first opioid consumption post-surgery; and worst and average NRS pain intensity scores at 24, 48, 72, and 96 hours post-surgery.

Safety endpoints included incidence of treatment emergent adverse events (AEs) and serious adverse events (SAEs) from start of the nerve block procedure through POD14.

Pharmacokinetic endpoints included area under the plasma concentration-versus-time curve (AUC); maximum plasma concentration (Cmax) and time of maximum plasma concentration (Tmax); apparent terminal elimination half-life (t_{1/2el}); apparent clearance (CL/F); and apparent volume of distribution (Vd).

Pharmacodynamic endpoints included: onset of sensory block, offset of sensory block, duration of sensory block, onset of motor block, offset of motor block, and duration of motor block.

TABLE 1
Pharmacokinetic Parameters

| Parameter | Definition |
|---|---|
| Cₘₐₓ | The maximum observed plasma concentration obtained directly from the concentration-time data |
| Early Cₘₐₓ¹ | The maximum observed plasma concentration occurring between dosing (0 h) and x h after |
| | dosing if appropriate based on the individual subject and treatment group mean concentration- |
| | time plots, where x was determined from the concentration-time plot |
| Late Cₘₐₓ¹ | The maximum observed plasma concentration occurring more than x h after dosing if appropriate |
| | based on the individual subject and treatment group mean concentration-time plots, where x was a |
| | subject-specific local minimum turning point in the concentration-time curve |
| Tₘₐₓ | Time at which Cₘₐₓ was observed |
| Early Tₘₐₓ¹ | Time at which early Cₘₐₓ was observed |
| Late Tₘₐₓ¹ | Time at which late Cₘₐₓ was observed |
| λ_{z} | The apparent terminal elimination rate constant was estimated at the terminal phase by linear |
| | regression after log-transformation of the concentrations |
| t_{1/2el} | The apparent terminal half-life was calculated as ln(2)/λ_{z} |
| AUC₀₋ₗₐₛₜ | The area under the plasma concentration-time curve from the time of dosing to the time of the last |
| | quantifiable concentration; calculated using the linear-up/log-down trapezoidal method |
| AUC_{0-∞} | The area under the plasma concentration-time curve from the time of dosing (zero) to infinity; |
| | calculated as the sum of AUC₀₋ₗₐₛₜ and residual area Cₜ/λ_{z} |
| AUCₑₓₜᵣ | Extrapolated area under the curve from time of last point above LLOQ to infinity, expressed as |
| | percentage of AUC_{0-∞}; calculated as (Cₜ/λ_{z})/AUC_{0-∞} × 100%. |
| CL/F | Apparent clearance was estimated as dose/AUC_{0-∞}, where dose = 133 mg and 266 mg for subjects |
| | receiving EXPAREL 133 mg and 266 mg, respectively, and 44 mg (50 mg × 0.886 [salt to free |
| | base conversion]) for subjects receiving 50 mg bupivacaine HCl. |
| V_{d}/F | Apparent volume of distribution was estimated as (CL/F)/λ_{z} |

Abbreviations: HCL = hydrochloride; LLOQ = lower limit of quantitation; SAP = Statistical Analysis Plan.
¹If a subject only received bupivacaine HCl, the plasma concentration-time curve was not expected to see an early and late phase separation.

### Statistical Analysis

Descriptive statistics (number of subjects, mean, SD, median, minimum, and maximum) are provided for continuous data. Tabulations (number and percentage of subjects) by category are provided for categorical data. Safety analyses are summarized descriptively by treatment arms. The total sample size for Part A and B was calculated based on the primary outcome measure of NRS pain intensity scores. A sample size of 80 subjects per study arm [1:1 randomization, 80 EXPAREL (EXPAREL 266 or EXPAREL 133), 80 Bupivacaine HCl] provides at least 85% power to detect a treatment difference of 110 units in the AUCs (SD=230) comparing the EXPAREL arm with the Bupivacaine HCl arm at a one-sided 0.025 significance level. All tests will be either one-sided on a significance level of 0.025 or two-sided on a significance level of 0.05.

The AUC of NRS pain intensity scores from 0-96 hours post-surgery were analyzed using the analysis of covariance (ANCOVA) model.

Total postsurgical opioid consumption in oral morphine equivalents (OMED) from 0 to 96 hours is analyzed using the ANCOVA model. Time to first postsurgical opioid medication is analyzed using the Kaplan-Meier survival method. Worst and average NRS pain intensity scores through 24 h, 48 h, 72 h, and 96 h from the end of surgery are summarized by treatment arm.

Adverse event verbatim terms are mapped to preferred terms and related system organ class using the Medical Dictionary for Regulatory Activities (MedDRA). Events that start prior to the start of study drug administration are identified in a by-subject listings. Incidence rates of TEAEs and the proportion of subject prematurely withdrawn from the study due to a TEAE is shown for each treatment arm. Incidence rates are also displayed for each treatment arm for TEAEs by severity and separately by relationship. Incidence rates of SAEs is shown for each treatment arm. All incidence rates are categorized and displayed by system organ class and preferred term.

### Results

### Efficacy

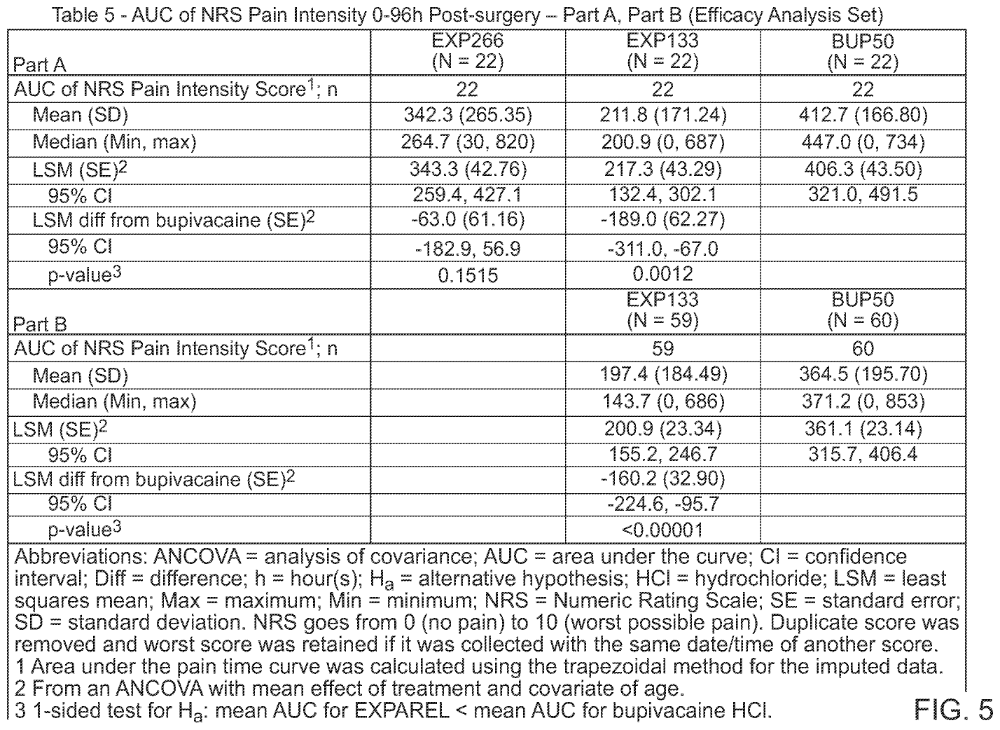

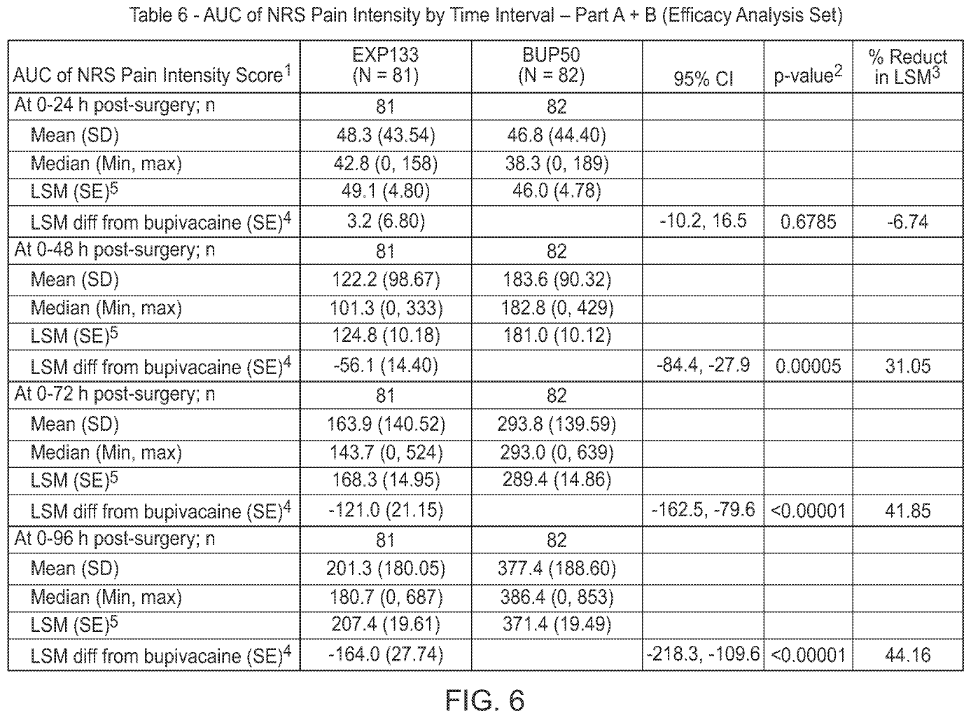

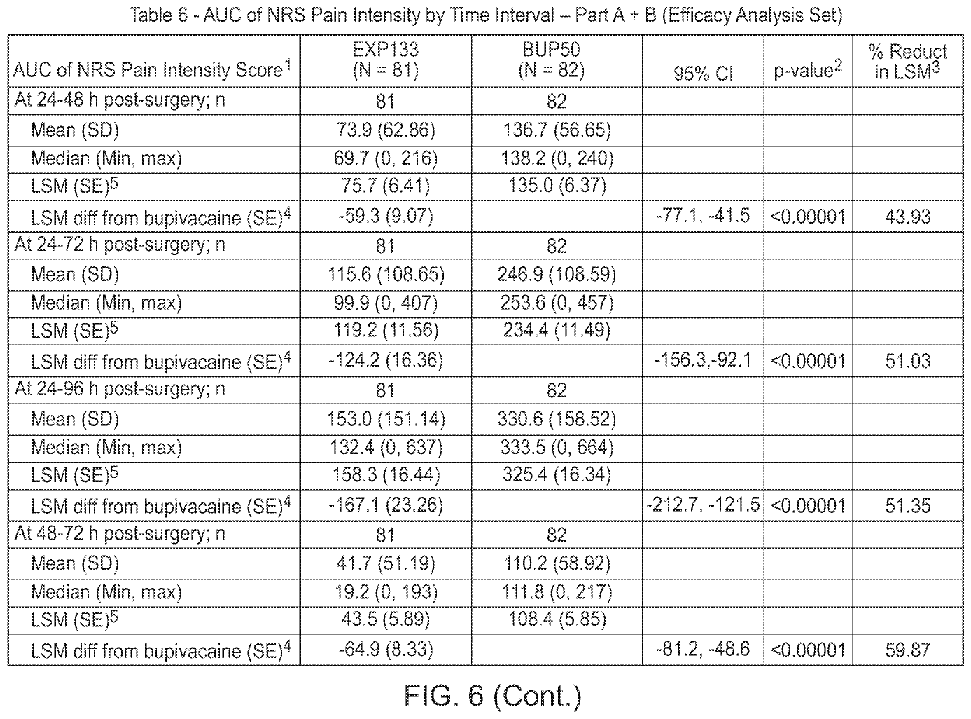

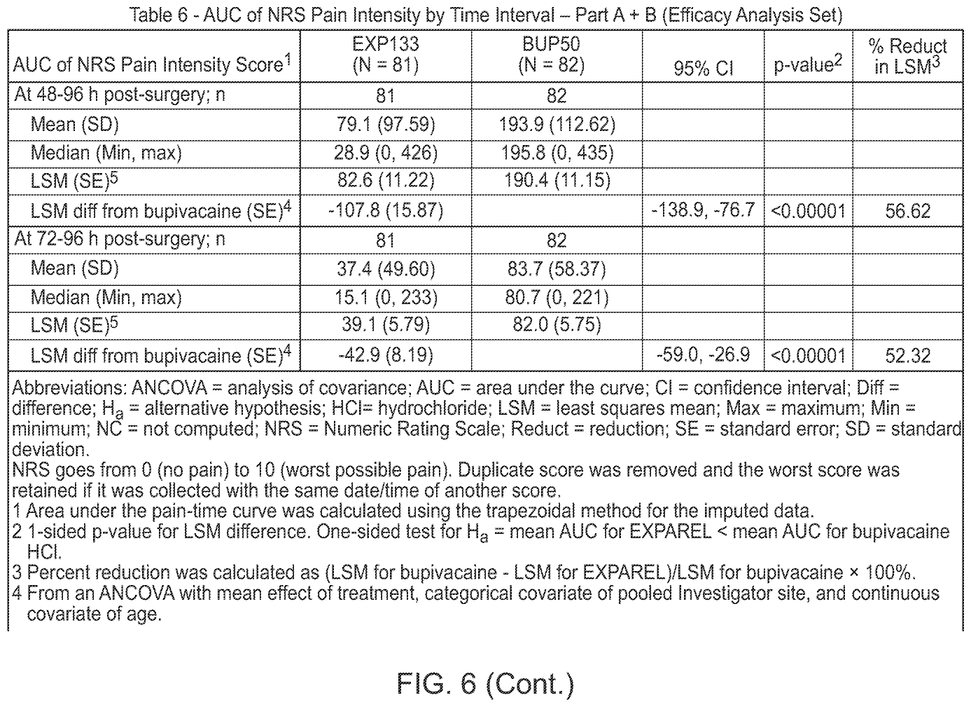

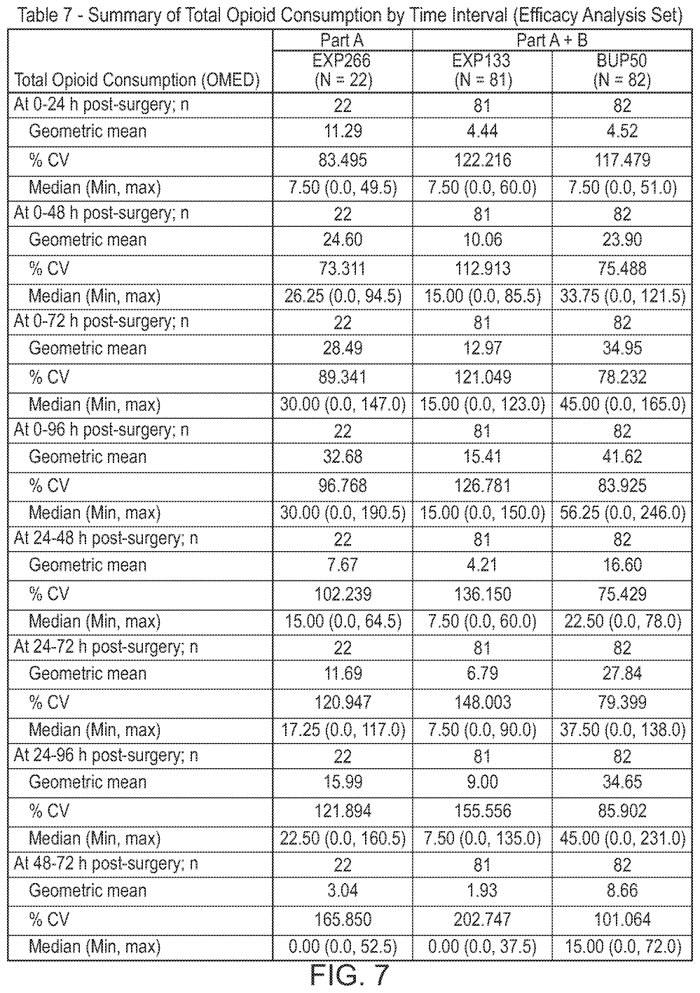

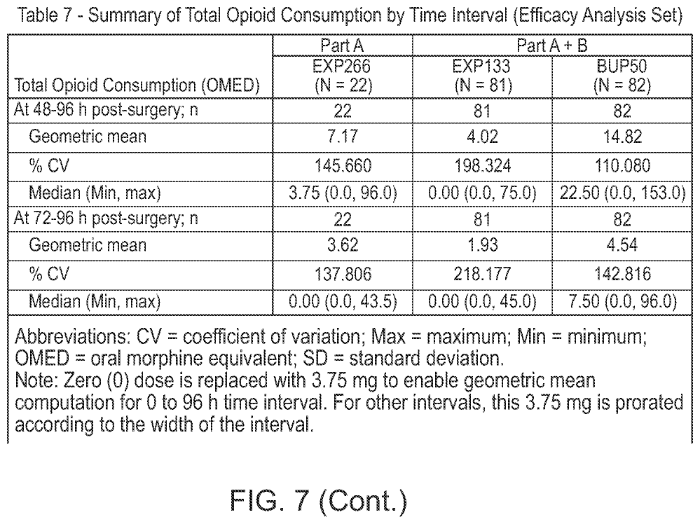

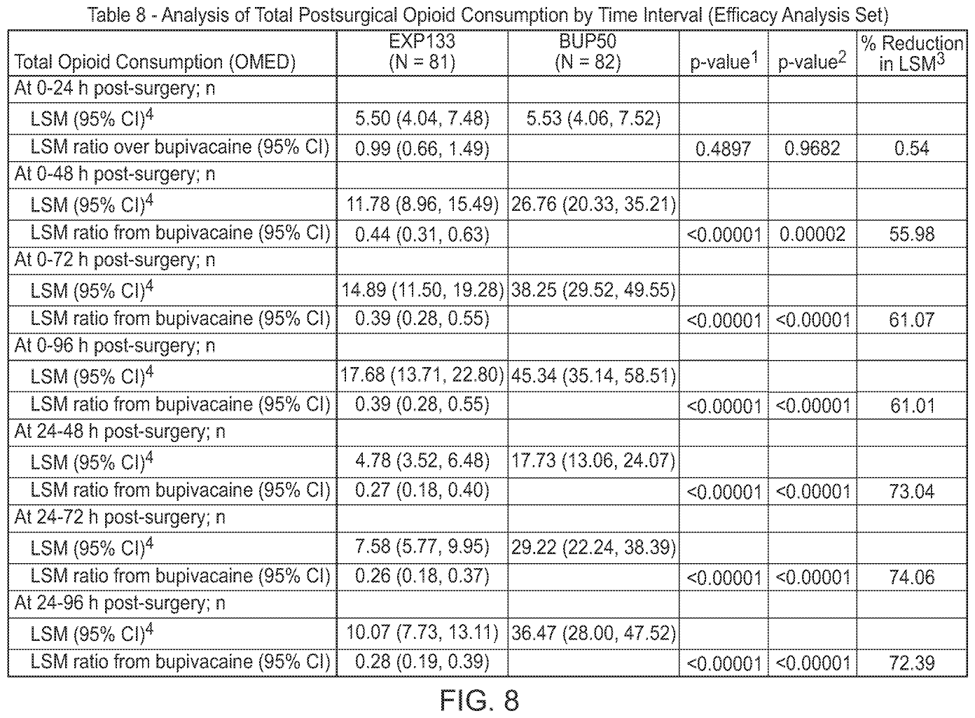

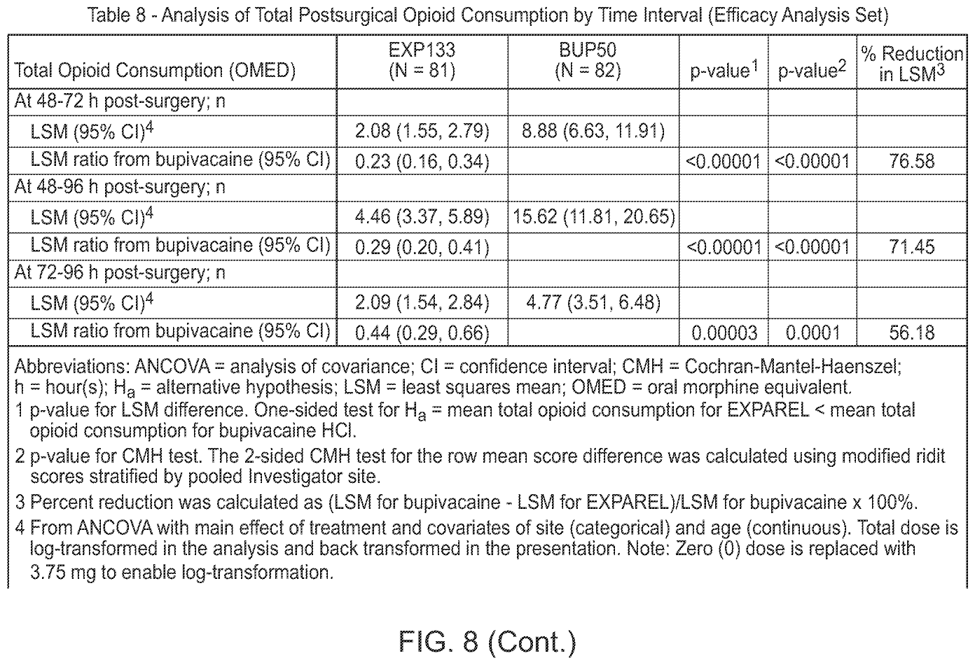

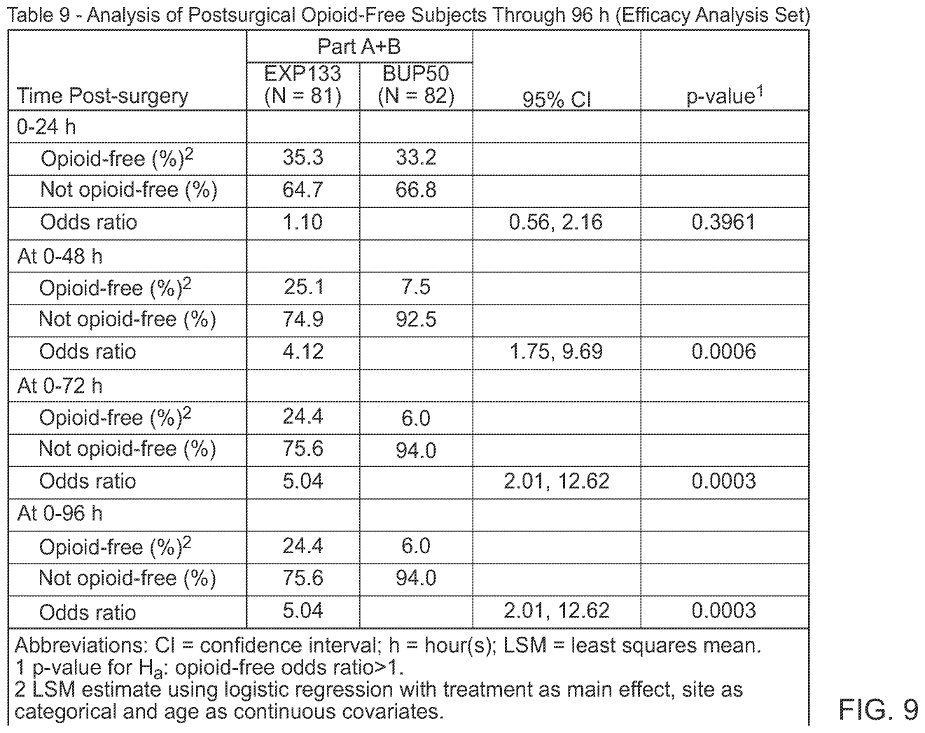

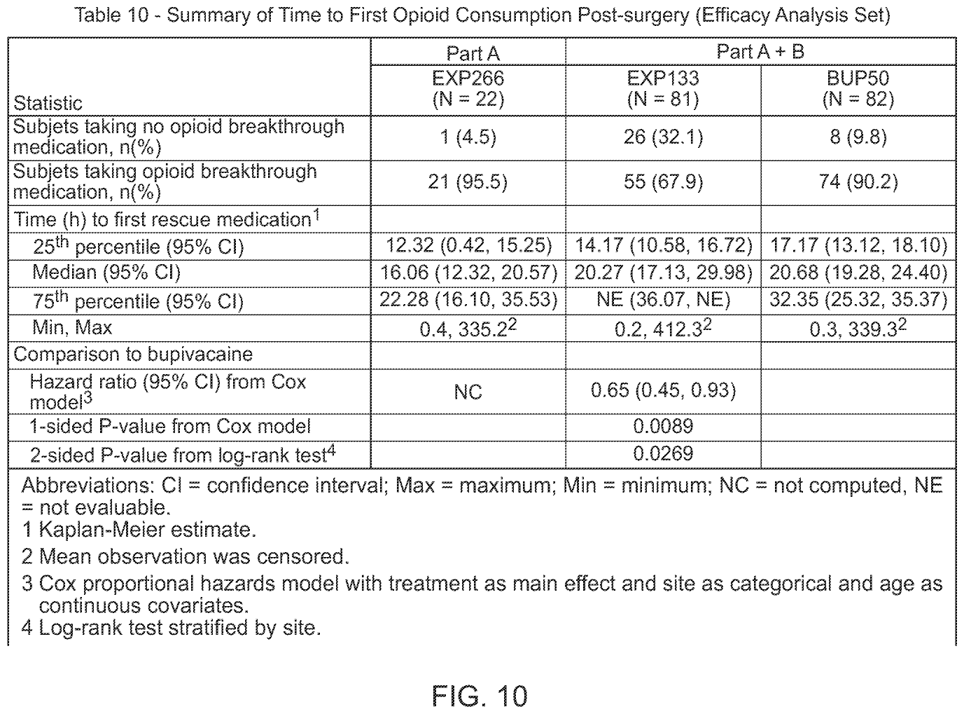

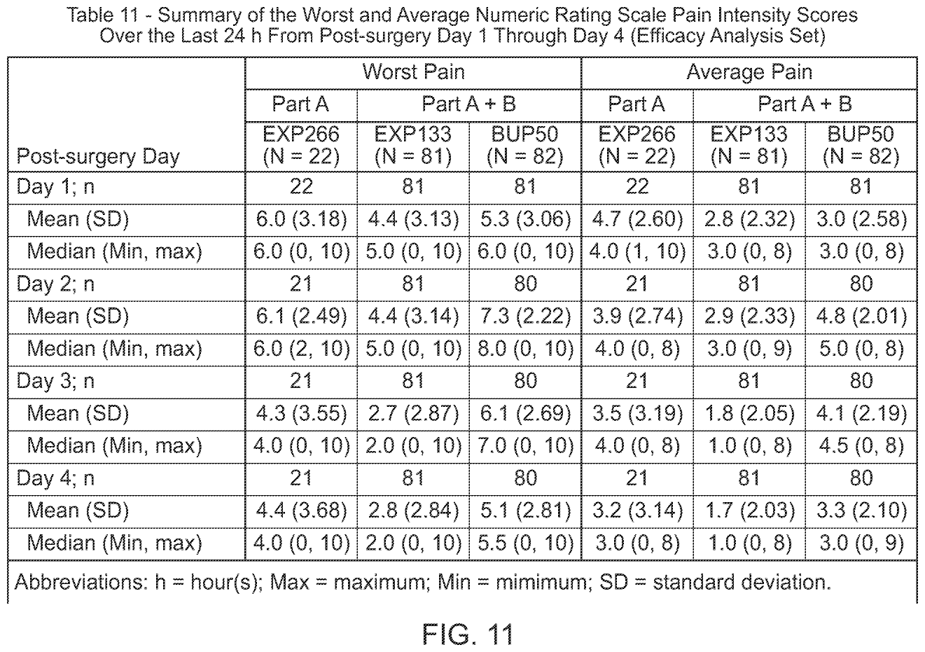

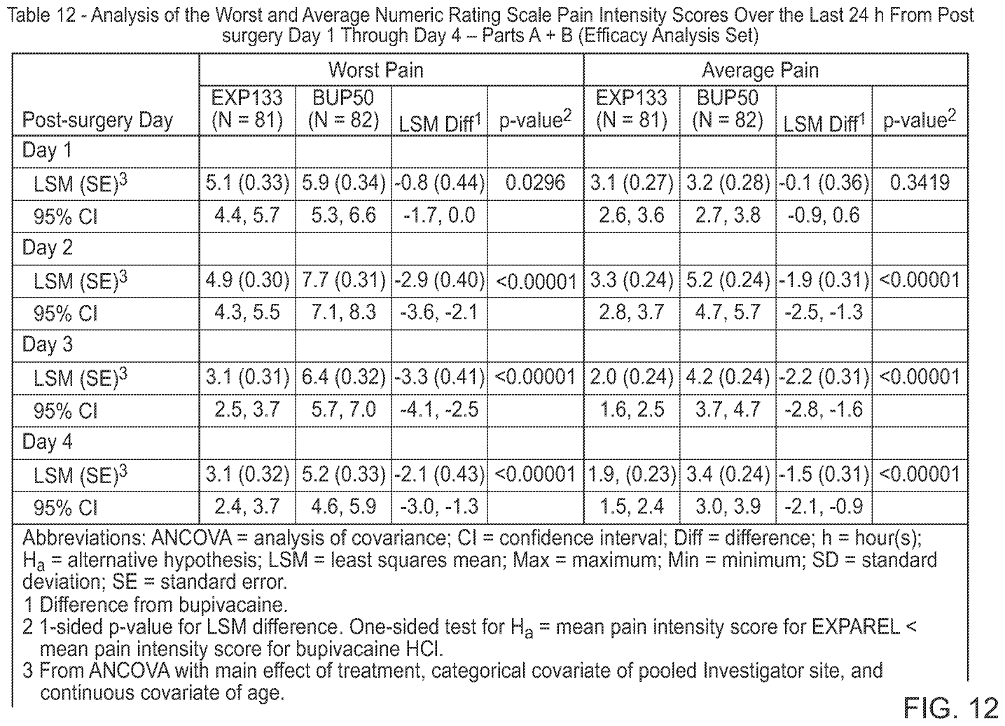

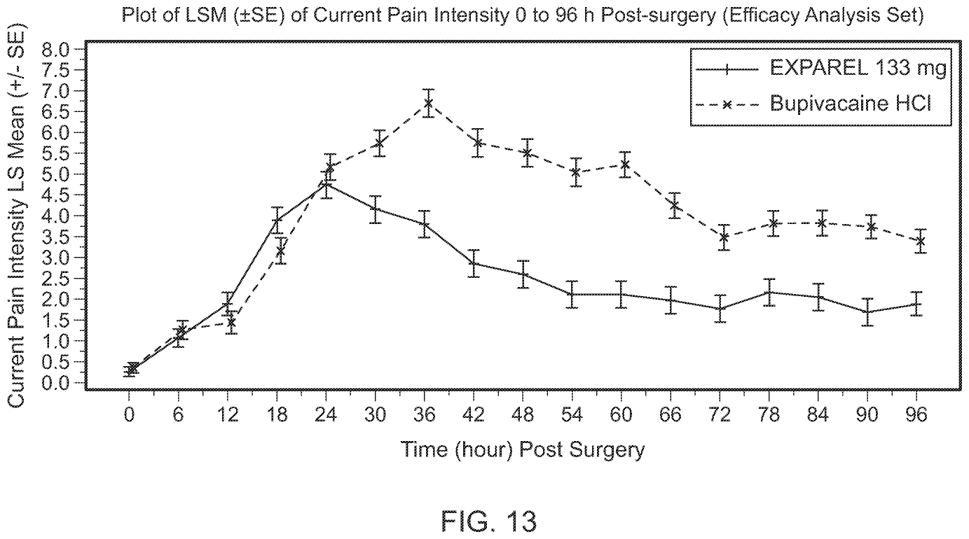

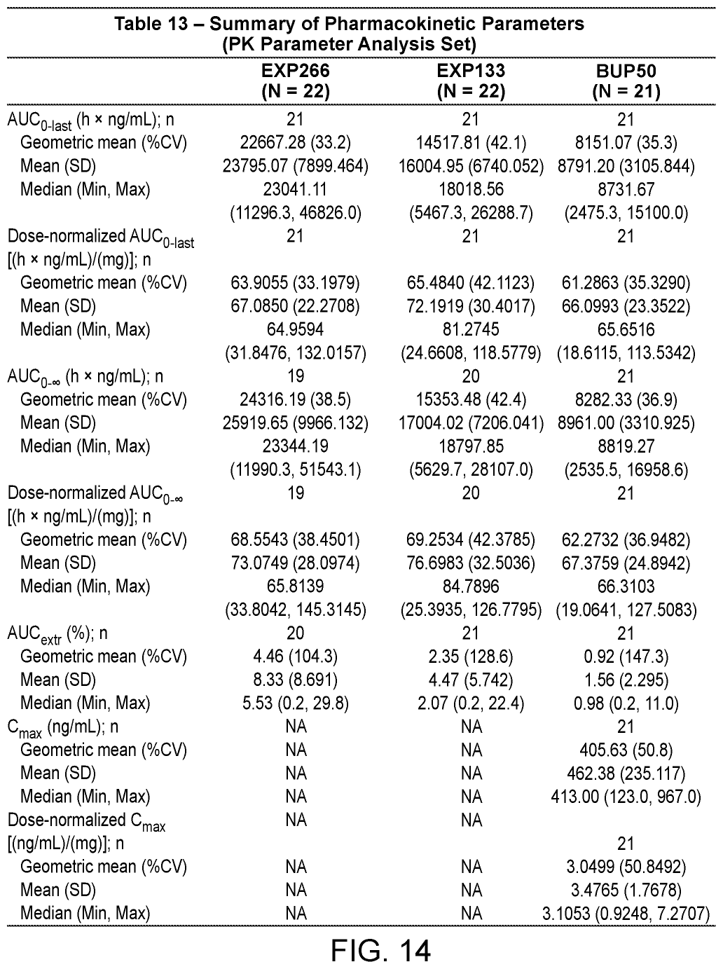

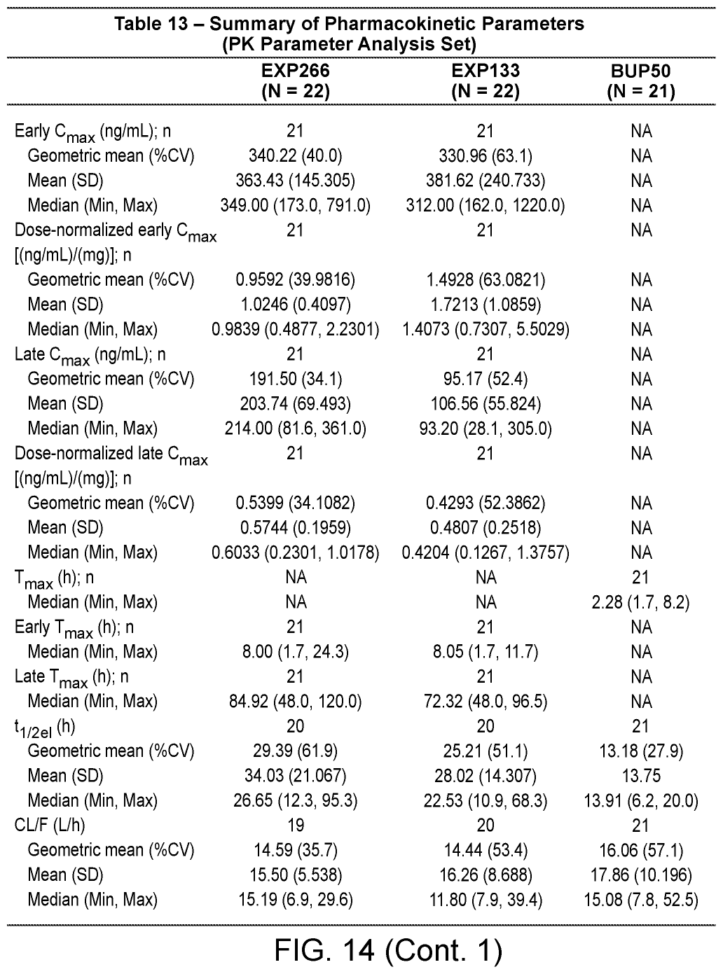

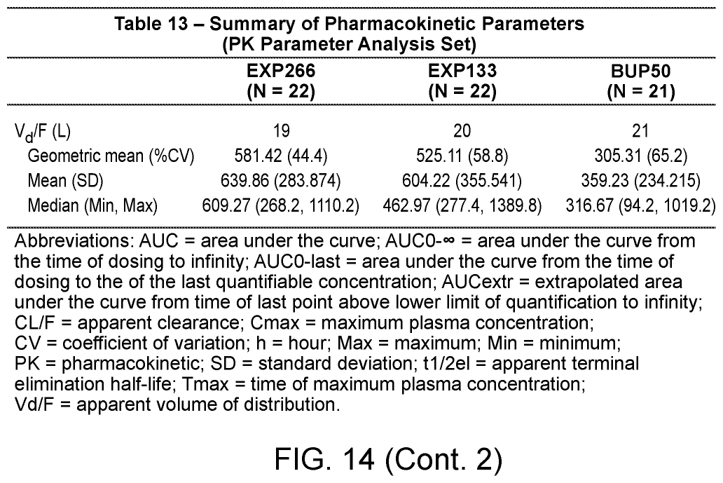

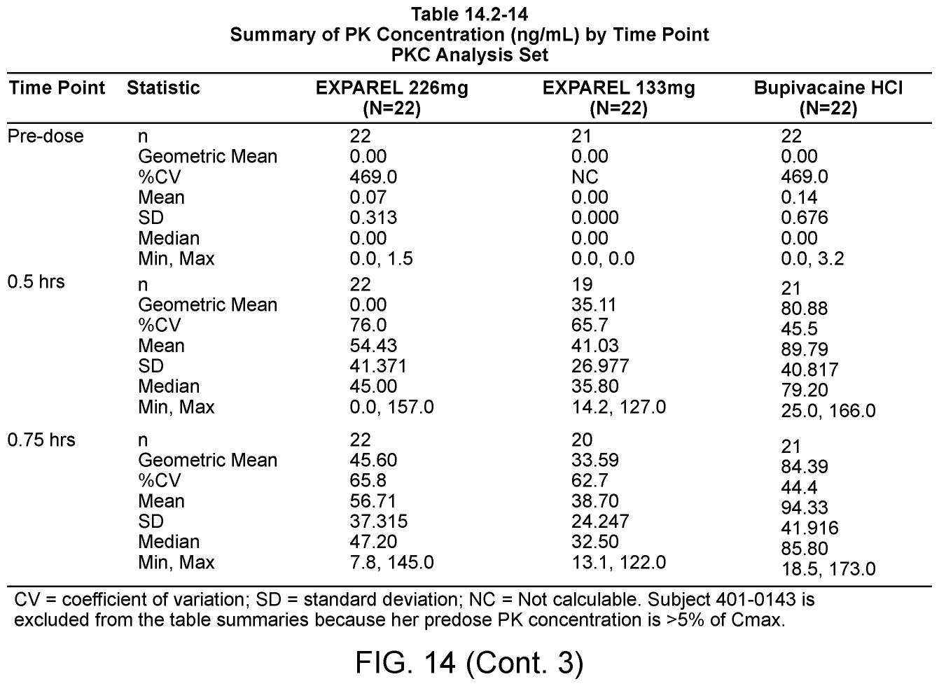

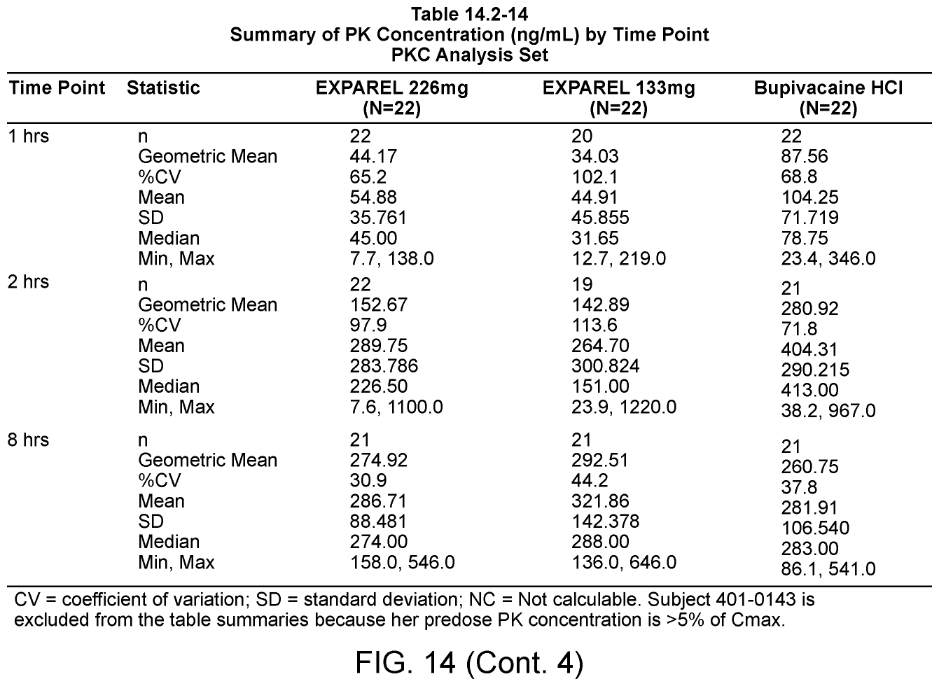

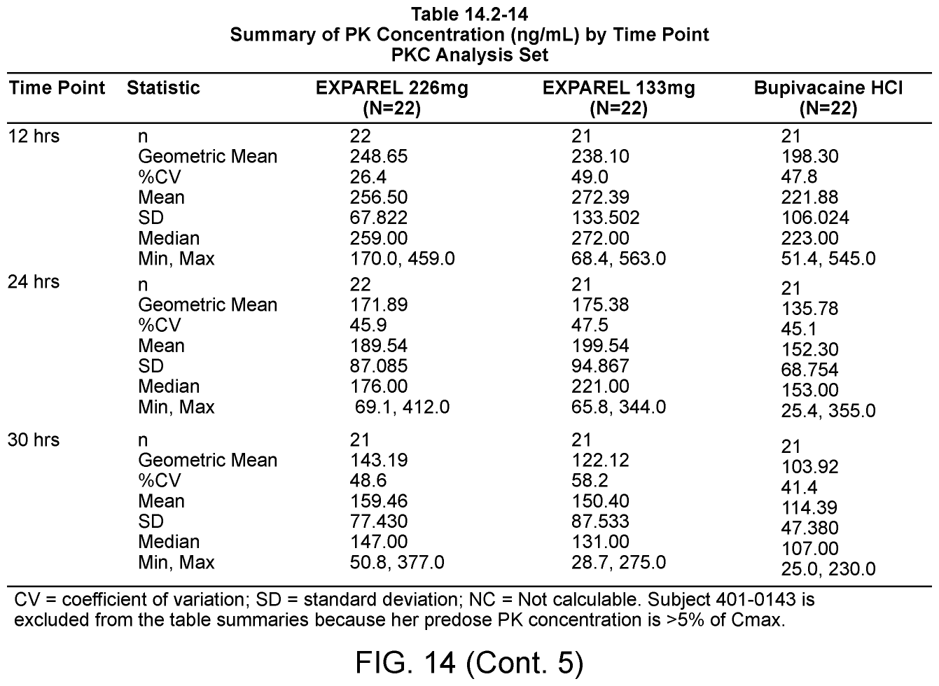

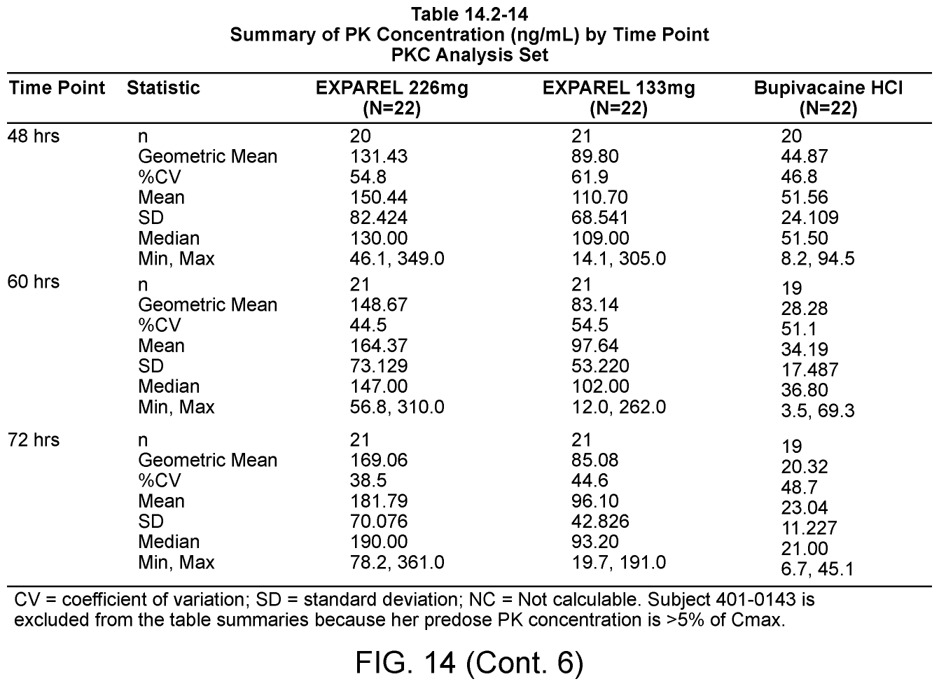

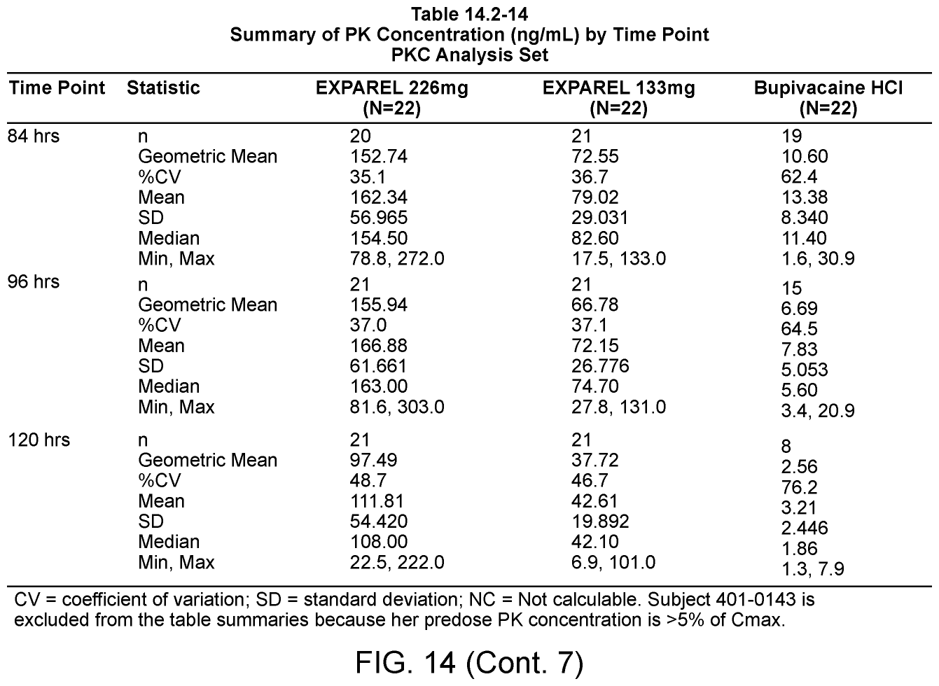

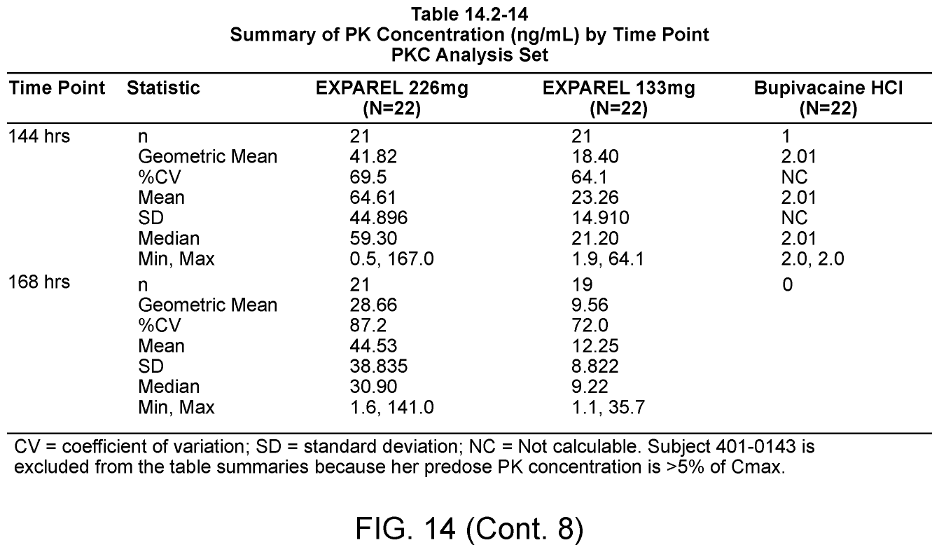

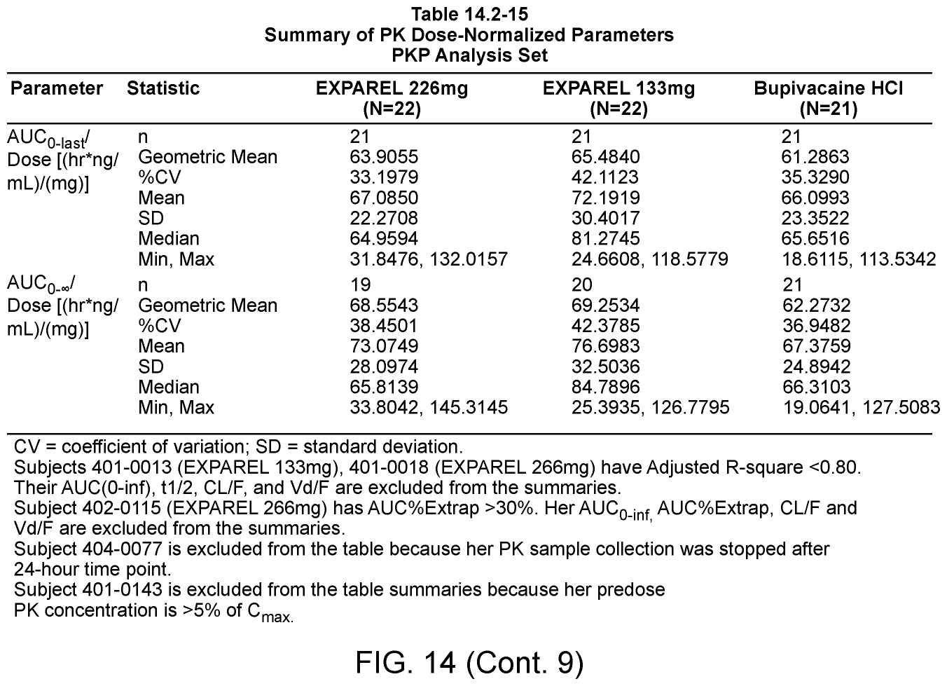

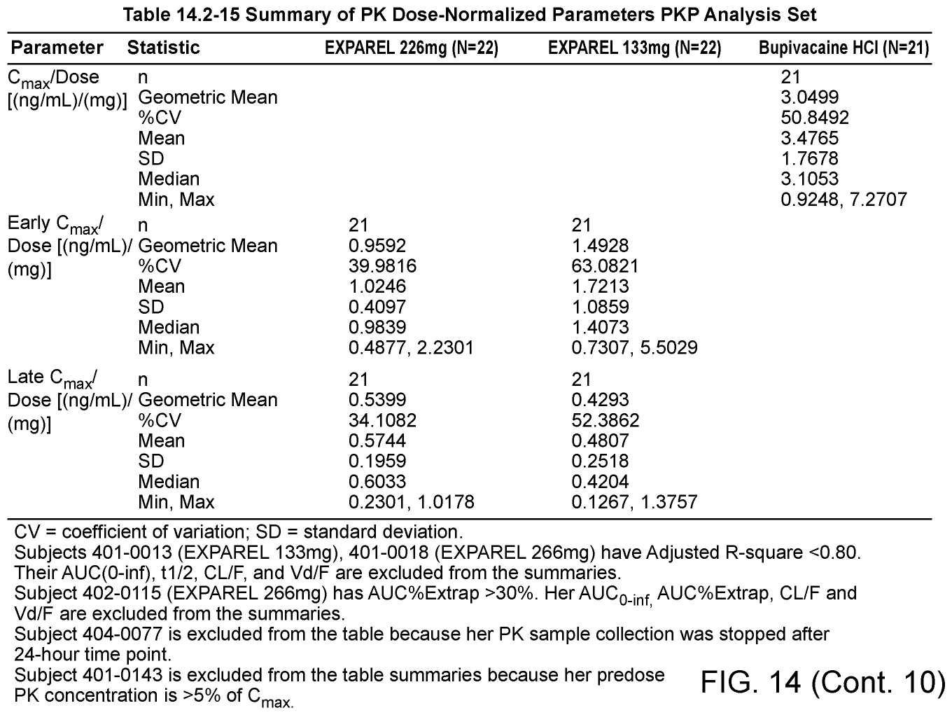

In Part A, there was a statistically significant difference in the LSM AUC of the NRS Pain Intensity Score from 0 to 96 h post-surgery between the EXP133 arm and BUP50 arm (LSM difference=−189.0; p=0.0012; FIG. 5); the difference was not significant between the EXP266 and BUP50 arm (p=0.1515; FIG. 5). In Part B, subjects in the EXP133 arm had a lower LSM AUC of NRS Pain Intensity Score from 0 to 96 h post-surgery compared with the BUP50 arm with an LSM difference of −160.2 (p<0.00001; FIG. 5). Similar results were observed when combining Part A and B subjects, with a corresponding 44.16% reduction in overall pain intensity for those in the EXP133 arm compared with the BUP50 arm (FIG. 6). After 24 h post-surgery, the EXP133 arm had significantly lower LSM AUC of NRS Pain Intensity Scores than the BUP50 arm overall (Part A+B) at all-time intervals (LSM difference range=−167.1 to −42.9; p values <0.00001; FIG. 6).
In Part A, the EXP133 arm had a geometric mean total opioid consumption approximately 2 fold lower than the EXP266 and BUP50 arms from 0 to 96 h post-surgery. Geometric mean total opioid consumption for EXP266 subjects in Part A was highest from 0 to 24 h post-surgery, decreasing over time at subsequent 24 h intervals, whereas EXP133 and BUP50 subjects had total opioid consumption peak at 24 to 48 h (Part A+B), before decreasing over time. Cumulatively, total opioid consumption was more than two-fold higher at 0 to 48 h (geometric mean range=4.44 to 11.29) compared with 0 to 24 h post-surgery (geometric mean range=10.06 to 24.60) in all treatment arms (FIG. 7). In Parts A+B combined, the EXP133 arm had a significantly lower total opioid consumption than the BUP50 arm with an LSM ratio of 0.39 from 0 to 96 h post-surgery (p<0.00001; FIG. 8). After 24 h post-surgery, the EXP133 arm had significantly lower total opioid consumption than the BUP50 arm at all-time intervals with LSM ratios over bupivacaine ranging from 0.23 to 0.44 (p values <0.00003; FIG. 8).
From 0 to 96 h post-surgery, estimates of the proportion of opioid-free subjects were 4.5% in the EXP266 arm (Part A), 33.3% in the EXP133 arm (Part A+B), and 9.8% in the BUP50 arm (Part A+B). Over the same time interval, subjects in the EXP133 arm were 5.04 times as likely to be opioid-free compared with the BUP50 arm (p=0.0003; FIG. 9).
Overall, the proportions of subjects who received opioid breakthrough medication were 95.5% in EXP266 (Part A), 67.9% in EXP133 (Part A+B), and 90.2% in BUP50 subjects (Part A+B). Median times to first rescue medication were 16.06 h in EXP266, 20.27 h in EXP133, and 20.68 h BUP50 subjects. In Parts A+B, the hazard ratio between EXP133 and BUP50 subjects was statistically significant at 0.65 (1 sided p value from Cox model=0.0089; FIG. 10).
Mean worst and average pain intensity scores were significantly lower in the EXP133 arm compared with the BUP50 arm on Days 2, 3, and 4 (LSM difference range=−3.3 to −2.1; p values <0.00001 and LSM difference range=−2.2 to −1.5; p-value <0.00001 for worst and average pain intensity, respectively; FIG. 12).
Estimate of opioid-free subjects were lower in EXP266 subjects (range=4.5 to 9.1%) compared with EXP133 (range=33.3 to 44.4%) and BUP50 (range=9.8 to 42.7%) subjects from Parts A and B at all-time intervals. Overall (Parts A+B), subjects in the EXP133 arm were 4.12 and 5.04 times as likely to be opioid free compared with the BUP50 arm at 0 to 48 h (p=0.0006) and 0 to 72 h post-surgery (p=0.0003), respectively (FIG. 9).
Subject satisfaction with pain management was similar amongst the 3 treatment groups with mean values between 9.0 and 9.3 out of 10.
Mean AUC of worst and average pain intensity in the EXP266 arm (Part A) was similar to the BUP50 arm (Part A+B), but both treatment arms were higher compared with the EXP133 arm (Part A+B). Mean AUC of worst pain over the last 24 h post-surgery from POD1 through POD4 was significantly different (LSM difference=9.1, p<0.00001) between the EXP133 and BUP50 arm with values of 16.1 and 25.2, respectively. Mean AUC of average pain over the last 24 h post-surgery from POD1 through POD4 was significantly different (LSM difference=−5.8, p<0.00001) between the EXP133 and BUP50 arm with values of 10.3 and 16.1, respectively.
Mean current pain intensity post-surgery peaked at 24 h post-surgery in the EXP133 arm, 30 h in the EXP266 arm, and 36 h in the BUP50 arm, gradually decreasing until 96 h post-surgery. From 30 to 96 h post-surgery (mean range=0.4 to 6.0 vs. mean range=0.1 to 4.2 in EXP133 [Part A+B] and 0.2 to 5.3 in BUP50 [Part A+B]), the EXP133 arm had a significantly lower mean current pain intensity than the BUP50 arm (p-values <0.00008; FIG. 13).

### Exploratory Efficacy Measures

Estimates of opioid-free subjects were lower in EXP266 subjects (range=4.5 to 9.1%) compared with EXP133 (range=33.3 to 44.4%) and BUP50 (range=9.8 to 42.7%) subjects from Parts A and B at all-time intervals. Overall (Part A+B), subjects in the EXP133 arm were 4.12 and 5.04 times as likely to be opioid-free compared with the BUP50 arm at 0 to 48 h (p=0.0006) and 0 to 72 h post-surgery (p=0.0003), respectively. See FIG. 9.

### Pharmacodynamics

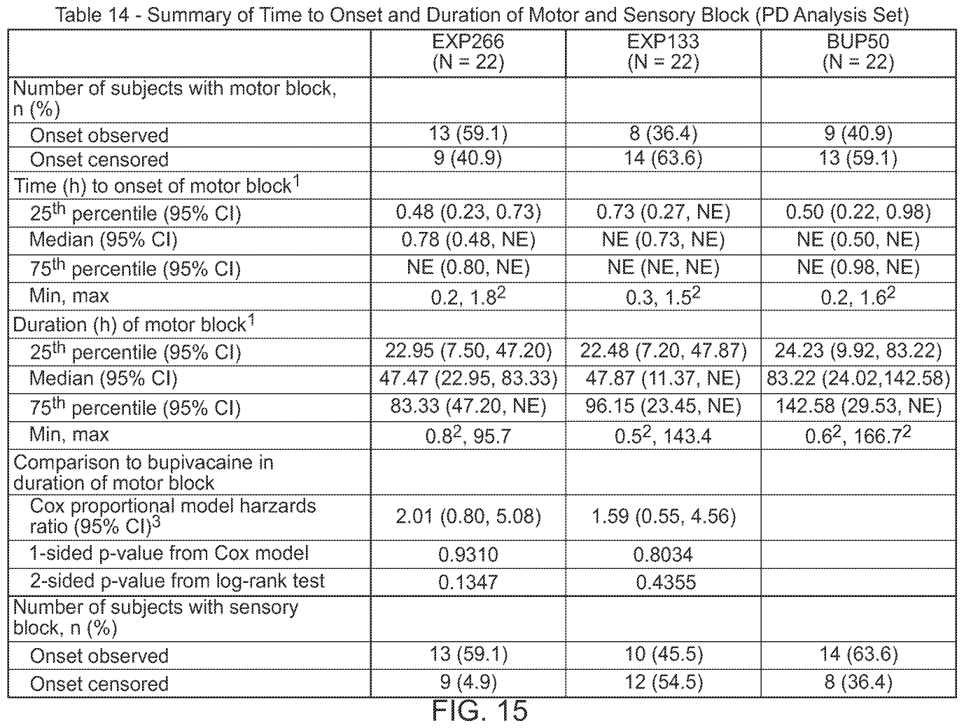

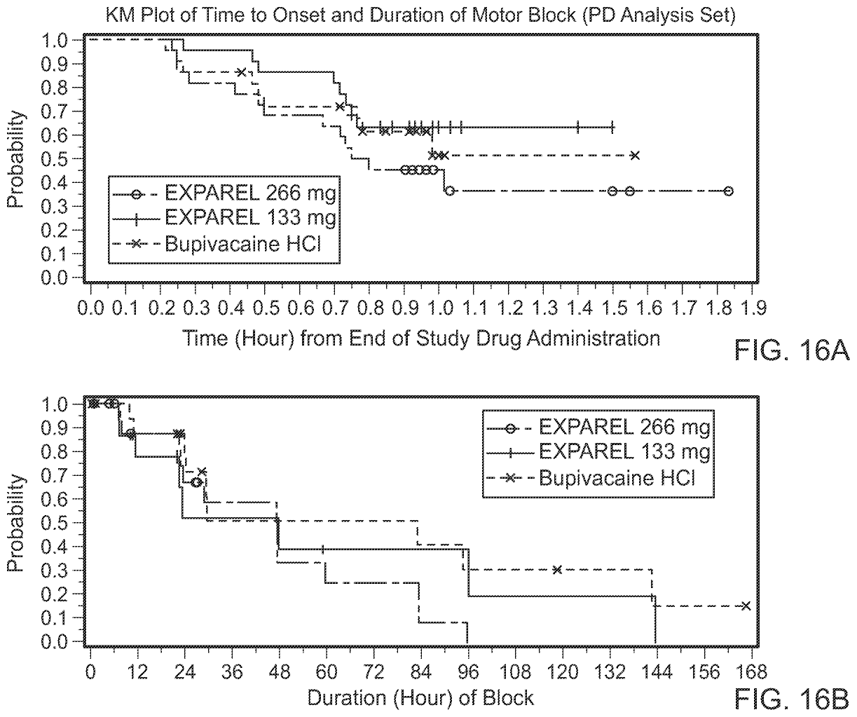

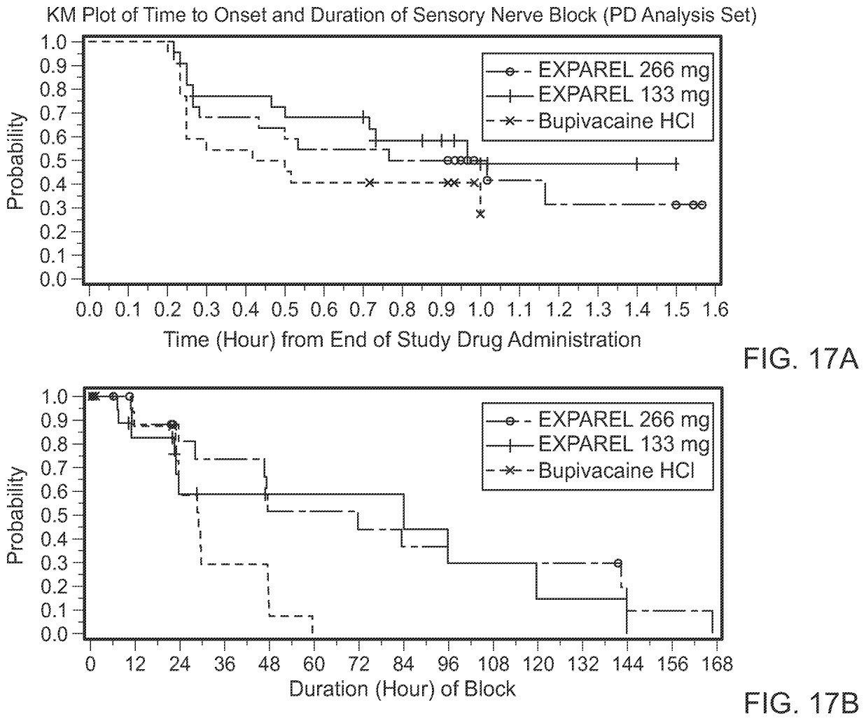

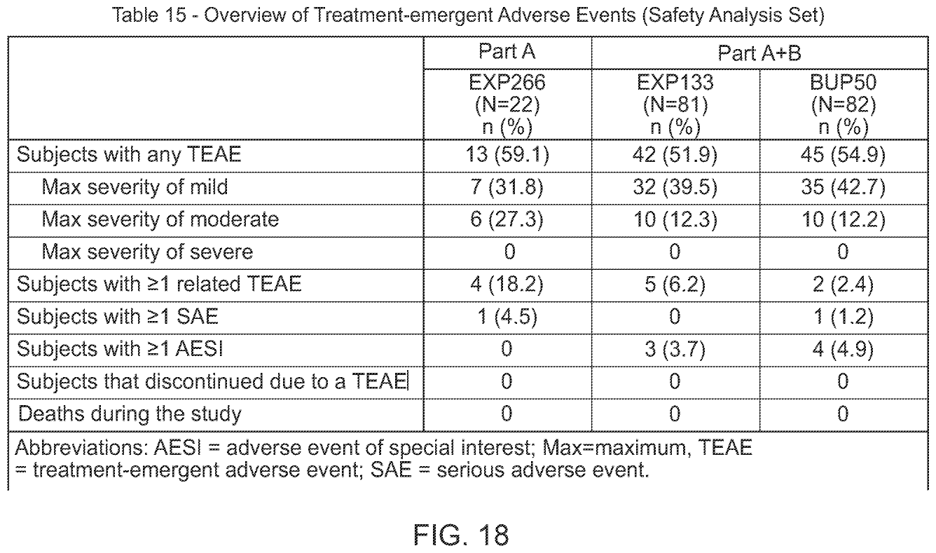

There were 59.1% of EXP266 subjects with motor block onset observed, compared with 36.4% and 40.9% of EXP133 and BUP50 subjects, respectively. The median time to motor block was not evaluable in all treatment arms. The difference between median duration of motor block was not statistically significant between treatment arms. The first time point at which foot movement was not present in ≥50% of subjects was 1 h post dose for the EXP266 arm (50.0%) and 2 h post-dose for the EXP133 and BUP50 arms (54.5% each). By 84 h post-dose, 18.2% of BUP50, 13.6% of EXP133, and 4.5% of EXP266 subjects still did not have complete foot movement (FIGS. 15 and 16).
There were 59.1% of EXP266 and 63.6% of BUP50 subjects with sensory block onset observed, compared with 45.5% of EXP133 subjects. The median time to sensory block was 0.46 h in BUP50, 0.89 h in EXP266, and 0.97 h in EXP133 subjects. The median duration of sensory block was 2- to 3 fold longer in EXPAREL subjects (71.87 h in EXP266 and 84.28 h in EXP133) compared with BUP50 subjects (29.10 h); these differences were only statistically significant for EXP266 subjects (1-sided p-value from Cox model=0.0069). The first time point at which sensation was not present in ≥50% of subjects was 30 min post-dose for the BUP50 arm (59.1%) and 2 h for the EXP266 (68.2%) and EXP133 (59.1%) arms. At 60 h post-dose, 40.9% of EXP266 and 18.2% of EXP133 subjects still did not have completion sensation; conversely, all BUP50 subjects had regained sensation (FIGS. 15 and 17).

### Safety

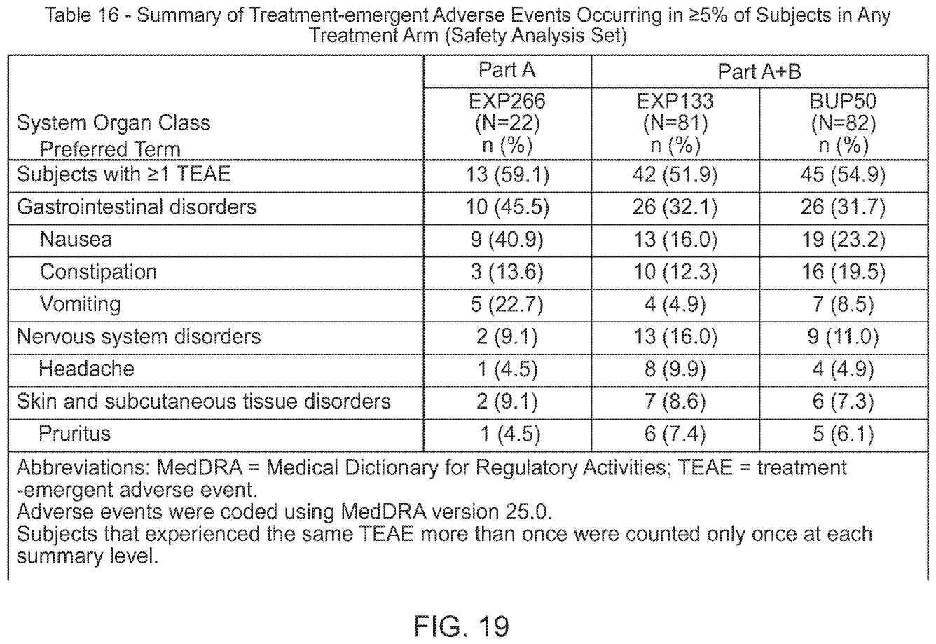

No deaths occurred during this study and EXPAREL was well tolerated by subjects.
A similar proportion of subjects across treatment groups reported TEAEs, which included 59.1% of EXP266 subjects, 54.9% of BUP50 subjects, and 51.9% of EXP133 subjects. The most common TEAEs were nausea (40.9%) and vomiting (22.7%) in EXP266 subjects, nausea (16.0%) and constipation (12.3%) in EXP133 subjects, and constipation (31.7%) and nausea (23.2%) in BUP50 subjects (FIG. 20).
A slightly higher proportion of subjects in the EXP266 arm had a treatment-related TEAE (Part A; 18.2%) compared with the EXP133 (Part A+B; 6.2%) and BUP50 (Part A+B; 2.4%) arms (FIG. 19). Hypertension and peroneal nerve palsy were the most common related events occurring in 3 and 2 subjects, respectively. Related TEAEs of moderate severity included hypertension, headache, vomiting, bradycardia, atrioventricular block second degree, and muscular weakness; all other events were of mild severity. The related event of arthralgia was assessed as probably related to study drug, while all other events were assessed as possibly related.
All TEAEs were of mild or moderate severity. Between 31.8% and 42.7% of subjects had a mild TEAE and 12.2% to 27.3% had a moderate TEAE across the treatment arms (FIG. 19). There was a higher proportion of subjects with moderate TEAEs in the EXP266 arm (27.3%) compared with 12.3% in the EXP133 arm and 12.2% in the BUP50 arm (FIG. 19). The most common moderate severity TEAEs were nausea, constipation, vomiting, and headache.
There were 3 subjects in the EXP133 arm and 4 subjects in the BUP50 arm who had ≥1 AESI, which included AESIs of muscle twitching, dizziness, peroneal nerve palsy, atrioventricular block second degree, and muscular weakness. Dizziness was the most common AESI, reported in 3 subjects. In the BUP50 arm, the AESIs of atrioventricular block second degree and muscular weakness were considered possibly related to study drug (1 subject each).
There was 1 subject in the EXP266 arm that had treatment-emergent SAEs of hypertension and pyrexia related to study drug and 1 subject in the BUP50 arm had an SAE of atrioventricular block second degree related to study drug; both subjects discontinued from the study due to SAEs. Two of the 3 SAEs (atrioventricular block second degree and hypertension) were of moderate severity and all SAEs resolved the same day as the start date. There were no other subjects with SAEs or TEAEs leading to study discontinuation.
Overall, there were minimal changes in mean vital sign values from baseline to post-surgery. Mean values also did not markedly differ across treatment arms. Mean systolic and diastolic blood pressure were generally lower following surgery across treatment arms.
From Screening to POD7, scheduled EKG assessments were normal or not clinically significant for most subjects, with 1 subject in the EXP133 and BUP50 arm having a clinically significant abnormal EKG finding on POD2 and POD1, respectively. Following study drug administration, clinically significant findings included 1 subject in the EXP133 arm with a sinus rhythm with poor R wave progression; 1 subject in the BUP50 arm had a clinically significant abnormality related to the SAE of second degree atrioventricular block.

### Summary of Results and Conclusions

Efficacy Results—Pain Intensity Scores: EXP133 subjects had a significantly lower LSM AUC of NRS Pain Intensity Score than the BUP50 arm from 0 to 96 h post-surgery (LSM difference=−164.0; p<0.00001). After 24 h post-surgery, the EXP133 arm had significantly lower LSM AUC of NRS Pain Intensity Scores than the BUP50 arm at all-time intervals (LSM difference range=−167.1 to −42.9; p values <0.00001). Mean worst and average pain intensity scores were significantly lower in the EXP133 arm compared with the BUP50 arm on Days 2, 3, and 4 (LSM difference range=−3.3 to −2.1; p values <0.00001 and LSM difference range=−2.2 to 1.5; p value <0.00001 for worst and average pain intensity, respectively).
Efficacy Results—Postsurgical Opioid Consumption: The EXP133 arm had a significantly lower total opioid consumption than the BUP50 arm from 0 to 96 h post-surgery (LSM ratio=0.39; p<0.00001). Over the same time interval, subjects in the EXP133 arm were 5.04 times as likely to be opioid-free compared with the BUP50 arm (p=0.0003). The median time to first rescue medication was 20.27 h and 20.68 h in EXP133 and BUP50 subjects, respectively.
Pharmacodynamic Results: The difference between median duration of motor block was not statistically significant between treatment arms; conversely, the median duration of sensory block was 2 to 3-fold longer in EXPAREL subjects (71.87 h in EXP266 and 84.28 h in EXP133) compared with BUP50 subjects (29.10 h).
Safety Results: No deaths occurred during this study and EXPAREL was well tolerated by subjects. A similar proportion of subjects across treatment groups reported TEAEs, which included 59.1% of EXP266 subjects, 54.9% of BUP50 subjects, and 51.9% of EXP133 subjects. There were 3 subjects in the EXP133 arm and 4 subjects in the BUP50 arm who had ≥1 AESI. Dizziness was the most common AESI, reported in 3 subjects. Five subjects (6.2%) and 2 (2.4%) subjects had ≥1 TEAE related to study drug in the EXP133 and BUP50 arms, respectively. There was 1 subject who received EXPAREL (266 mg) and 1 subject who received bupivacaine HCl that had SAEs.

### Adverse Events

An AE can be defined as any untoward medical occurrence associated with the use of a drug in humans, whether or not considered drug related. An AE (also referred to as an adverse experience) could be any unfavorable and unintended sign (e.g., abnormal laboratory finding), symptom, or disease temporally associated with the use of a drug, without any judgment about causality. An AE could arise from any use of the drug (e.g., off-label use in combination with another drug) and from any route of administration, formulation, or dose, including an overdose.

An AE could be any unfavorable and unintended change in a body structure or body function. Adverse events included any clinically significant deterioration of a subject's medical status. The AE could involve any organ or system and could be represented by the new onset or deterioration of a disease, syndrome, symptom, or physical sign, as well as by findings and results of instrumental examinations and laboratory tests. Any medically relevant and untoward change after the subject signed the ICF, including frequency or pattern changes for a fluctuating condition (e.g., migraine), was considered an AE.

An AE that occurred after administration of the study treatment was considered a treatment-emergent adverse event (TEAE). A continuous AE with varying grades of severity was to be recorded as 1 AE. The highest grade of severity experienced by that subject during the course of the continuous AE was to be recorded.

In general, the severity of an AE was to be categorized using the following guidelines:
Mild: An AE that was easily tolerated by the subject, caused minimal discomfort, and did not interfere with everyday activities.
Moderate: An AE that was discomforting and interfered with normal everyday activities.
Severe: An AE that prevented normal everyday activities.

Assessment of the relationship of the AE to study drug after careful medical consideration on a case-by-case basis. General guidelines are provided below:
Unrelated: A causal relationship between the study drug and the AE could be easily ruled out (e.g., based on the temporal sequence, absence of a reasonable pathophysiological mechanism, or direct evidence of actual cause).
Unlikely: A clinical event with a temporal relationship to study drug administration that made a causal relationship improbable and in which other drugs, chemicals, or underlying disease provided a plausible explanation.
Possible: A clinical event with a reasonable time sequence to administration of the study drug but that could also be explained by a concurrent disease or other drugs or chemicals.
Probable: A clinical event with a reasonable time sequence to administration of the study drug unlikely to be attributed to a concurrent disease or other drugs or chemicals and that followed a clinically reasonable response on withdrawal (dechallenge).
Definite: The pharmacological properties of the study drug(s) or of the substance class, and the course of the AE after dechallenge and, if applicable, after rechallenge, and/or specific test indicated involvement of the study drug(s) in the occurrence/worsening of the AE, and no indication of other causes existed.

Based on review of all peripheral nerve blocks, the following conditions were considered to be Adverse Events of Special Interest (AESIs) upon review of the AEs:
Falls
Persistent tingling
Persistent numbness
Persistent weakness
Hypersensitivity
Seizures
Tremors
Dizziness
Hematoma formation
Cardiovascular depression
Dyspnea
Cardiovascular arrest
Altered sensorium
Visual disturbances
Local anesthetic systemic toxicity

Adverse events of special interest classified as persistent referred to any condition (e.g., tingling, numbness, or sensory/motor weakness that affected the nerve block region, after the study drug administration) that persisted for >168 h from the time of onset.

A Serious Adverse Event (SAE) was defined as an AE or suspected adverse reaction that, in the view of either the Investigator or Sponsor, resulted in any of the following outcomes:
Death: Any event that resulted in a subject's death was reported as an SAE. However, death, in and of itself, was not an AE; it was an outcome. The cause of death was the AE.

Therefore, the Investigator was to make every effort to obtain and document the cause of death for all subjects who died during the study. If, despite all efforts, the cause of death remained unknown, the AE was to be documented as an “unspecified fatal event.”
Life-threatening: An AE was considered life-threatening if, in the view of either the Investigator or Sponsor, its occurrence placed the subject at immediate risk of death. It did not include an AE that, had it occurred in a more severe form, might have caused death.

Inpatient hospitalization or prolongation of existing hospitalization: Hospitalization, in and of itself, did not represent an SAE. It was the AE leading to the subject's hospitalization that became “serious” when it required inpatient care. Consequently, an SAE was not to be reported in case of preplanned hospitalizations for a pre-existing condition that did not worsen during the study. However, any medical condition that delayed a subject's discharge from the hospital (i.e., prolonged hospitalization) or required the subject to be readmitted was reported as an SAE.
Persistent or significant incapacity: A substantial disruption of a person's ability to conduct normal life functions.
Congenital anomaly/birth defect: If suspected that exposure to the study drug prior to conception or during pregnancy may have resulted in an adverse outcome in the child.
Medically significant: Important medical events that did not result in death, were not life-threatening, or did not require hospitalization, could be considered serious when, based upon appropriate medical judgment, they could jeopardize the subject and might have required medical or surgical intervention to prevent one of the outcomes listed in this definition.

Any SAE or death that occurred at any time after the subject signed the ICF through POD14, whether or not related to study drug, was to be reported within 24 h of discovery.

1. A method of administering regional analgesia comprising a sciatic nerve block in the popliteal fossa, the method comprising:
administering to a sciatic nerve of the patient via an injection needle 133 mg of a pharmaceutical composition;
wherein the pharmaceutical composition comprises multivesicular liposomes comprising: at least one amphipathic lipid, at least one neutral lipid, and bupivacaine phosphate, wherein the bupivacaine phosphate is encapsulated within the multivesicular liposome,
thereby administering the regional analgesia comprising the sciatic nerve block in the popliteal fossa.

2. The method of claim 1, wherein the administration comprises inserting the injection needle at an entry point in a leg of the patient.

3. The method of claim 1, wherein the regional analgesia is administered in connection with a bunionectomy.

4. The method of claim 2, wherein the entry point for the injection needle is the lateral thigh.

5. The method of claim 1, wherein the insertion of the injection needle into the leg of the patient comprises piercing the sciatic nerve sheath.

6. (canceled)

7. The method of claim 1, wherein the method comprises administering about 30 mL of the pharmaceutical composition.

8. The method of claim 1, wherein the multivesicular liposomes comprise:
bupivacaine or a salt thereof;
phosphoric acid;
a lipid component comprising at least one amphipathic lipid and at least one neutral lipid lacking a hydrophilic head group; and,
optionally, a cholesterol and/or a plant sterol,
wherein said multivesicular liposomes are made by a process comprising:
a) preparing a first aqueous component comprising phosphoric acid;
b) preparing a lipid component comprising at least one organic solvent, at least one amphipathic lipid, and at least one neutral lipid lacking a hydrophilic head group;
c) mixing said first aqueous component and said lipid component to form a water-in-oil emulsion, wherein at least one component comprises bupivacaine or a salt thereof;
d) mixing said water-in-oil emulsion with a second aqueous component to form solvent spherules; and
e) removing the organic solvent from the solvent spherules to form multivesicular liposomes encapsulating bupivacaine phosphate.

9. (canceled)

10. A method of administering to a peroneal and a tibial nerve of a human patient a pharmaceutical composition for post-operative analgesia, the method comprising:
administering via an injection needle 133 mg of a pharmaceutical composition to the popliteal fossa of the patient;
wherein the pharmaceutical composition comprises multivesicular liposomes comprising: at least one amphipathic lipid, at least one neutral lipid, and bupivacaine phosphate, wherein the bupivacaine phosphate is encapsulated within the multivesicular liposome,
thereby administering to the peroneal and the tibial nerve of the human patient the pharmaceutical composition for post-operative analgesia.

11. The method of claim 10, wherein the administration comprises inserting the injection needle at an entry point in a leg of the human patient.

12. The method of claim 11, wherein the entry point of the injection needle is the lateral thigh of the patient.

13. The method of claim 10, wherein the pharmaceutical composition is administered in connection with a bunionectomy.

14. The method of claim 11, wherein the insertion of the injection needle into the leg of the patient comprises piercing the sciatic nerve sheath.

15. The method of claim 10, wherein the method comprises administering about 30 mL of the pharmaceutical composition.

16. The method of claim 10, wherein the multivesicular liposomes comprise:
bupivacaine or a salt thereof;
phosphoric acid;
a lipid component comprising at least one amphipathic lipid and at least one neutral lipid lacking a hydrophilic head group; and,
optionally, a cholesterol and/or a plant sterol,
wherein said multivesicular liposomes are made by a process comprising:
a) preparing a first aqueous component comprising phosphoric acid;
b) preparing a lipid component comprising at least one organic solvent, at least one amphipathic lipid, and at least one neutral lipid lacking a hydrophilic head group;
c) mixing said first aqueous component and said lipid component to form a water-in-oil emulsion, wherein at least one component comprises bupivacaine or a salt thereof;
d) mixing said water-in-oil emulsion with a second aqueous component to form solvent spherules; and
e) removing the organic solvent from the solvent spherules to form multivesicular liposomes encapsulating bupivacaine phosphate.

17. (canceled)

18. A method of treating post-operative foot pain in a patient, the method comprising:
administering to the patient 133 mg of a pharmaceutical composition to a sciatic nerve;
wherein the pharmaceutical composition comprises multivesicular liposomes comprising: at least one amphipathic lipid, at least one neutral lipid, and bupivacaine phosphate, wherein the bupivacaine phosphate is encapsulated within the multivesicular liposome,
thereby treating post-operative foot pain in the patient.

19. The method of claim 18, wherein the administration comprises inserting the injection needle at an entry point in a leg of the human patient.

20. The method of claim 19, wherein the entry point of the injection needle is into a lateral thigh of the patient.

21. The method of claim 18, wherein the pharmaceutical composition is administered in connection with a bunionectomy.

22. (canceled)

23. The method of claim 18, wherein the insertion of the injection needle into the leg of the patient comprises piercing the sciatic nerve sheath.

24. The method of claim 18, wherein the method comprises administering about 30 mL of the pharmaceutical composition.

25. The method of claim 18, wherein the multivesicular liposomes comprise:
bupivacaine or a salt thereof;
phosphoric acid;
a lipid component comprising at least one amphipathic lipid and at least one neutral lipid lacking a hydrophilic head group; and,
optionally, a cholesterol and/or a plant sterol,
wherein said multivesicular liposomes are made by a process comprising:
a) preparing a first aqueous component comprising phosphoric acid;
b) preparing a lipid component comprising at least one organic solvent, at least one amphipathic lipid, and at least one neutral lipid lacking a hydrophilic head group;
c) mixing said first aqueous component and said lipid component to form a water-in-oil emulsion, wherein at least one component comprises bupivacaine or a salt thereof;
d) mixing said water-in-oil emulsion with a second aqueous component to form solvent spherules; and
e) removing the organic solvent from the solvent spherules to form multivesicular liposomes encapsulating bupivacaine phosphate.

26. (canceled)

27. (canceled)

28. (canceled)

29. (canceled)

30. (canceled)
